(19) United States
(12) Patent Application Publication
(10) Pub. No.: US 2015/0239824 A1
(43) Pub. Date: Aug. 27, 2015

(54) BRANCHED OR MACROCYCLIC POLYAMINES AND USES THEREOF

(71) Applicants: UNIVERSITE DE STRASBOURG, STRASBOURG (FR); CENTRE NATIONAL DE LA RECHERCHE SCIENTIFIQUE, PARIS (FR)

(72) Inventors: Marie-France Carlier, Gif Sur Yvette (FR); Girish Kumar Koripelly, Bangalore (IN); Jean-Marie Lehn, Strasbourg (FR); Iliana Nedeva, Gabrovo (BG); Daniel Riveline, Strasbourg (FR)

(21) Appl. No.: 14/423,533

(22) PCT Filed: Aug. 23, 2013
(86) PCT No.: PCT/EP2013/067589

§ 371 Date: Feb. 24, 2015

Related U.S. Application Data
(60) Provisional application No. 61/692,809, filed on Aug. 24, 2012.

(30) Foreign Application Priority Data
Jul. 26, 2013 (EP) 13306088.9

(51) Int. Cl. C07C 211/14; C07D 259/00; G01N 33/50; C07D 209/14

(52) U.S. Cl. CPC C07C 211/14; C07D 209/14; C07D 259/00; G01N 33/5026

(57) ABSTRACT

The present invention relates to synthetic polyamines and their polyammonium derivatives and their use as a pharmacological or biological research tool or as a therapeutic agent.

The present invention is concerned with synthetic polyamines, their polyammonium derivatives and their use as pharmacological or biological research tools or as therapeutic agents

### BACKGROUND OF THE INVENTION

The cytoskeleton provides the cell with structure and shape and plays important roles in intracellular transport and cell division. The cytoskeleton is also involved in key steps of embryogenesis and other cellular processes. For instance, the cytoskeleton may form structures such as cilia, flagella and lamellipodia involved in cell motility.

In eukaryotic cells, the cytoskeleton is composed of three kinds of cytoskeletal filaments, namely microtubules, microfilaments and intermediate filaments. Microfilaments are the main components of cell cytoskeleton and are generally organized into lamellar networks and bundles. Microfilaments consist in linear polymers of actin monomers that spontaneously self-assemble and disassemble, whereby the micro filaments take part in cell migration and cell division. On the other hand, microtubules are polymers of tubulin and play pivotal roles in a number of cellular processes such as intracellular transport of substances, intracellular movement of organelles and cell division.

The cytoskeleton is thus a complex network of polymers in dynamic assembly, which continuously remodels itself It is apparent that any dysfunction of cytoskeleton dynamics assembly may contribute to the onset or the development of disorders and diseases. For instance, the onsets of certain metastasis and neurodegenerative disorders were shown to be associated with dysfunction of the cytoskeleton.

A better understanding of cytoskeleton dynamics, and in particular those of microfilaments, is thus crucial to elucidate processes involved in a plurality of cell events. Cytoskeleton is also a potential therapeutic target for a variety of diseases. Thus, identifying new molecules able to target the cytoskeleton is a major challenge in pharmacology.

Until now, available cytoskeletal drugs are quite limited. Most of them target tubulin or actin and mainly act either as microtubules or microfilaments stabilisers or as inhibitors of tubulin or actin polymerization. Examples of cytoskeletal drugs targeting tubulin include certain chemotherapeutic agents such as colchicine, vinblastine, vincristine and nocodazole, which prevent the polymerization of microtubules, or taxol and docetaxel which stabilize microtubules. On the other hand, drugs able to impair actin dynamics are also known. For instance, latrunculin A and B are known to promote filament depolymerization by sequestering G-actin; cytochalasin D inhibits actin polymerization; jasplakinolide stabilizes filaments and blocks assembly dynamics. Finally, phalloidin prevents the depolymerization of actin filaments and is mainly used as an imaging tool for investigating actin filaments in cells.

More recently, screening experiments enabled to identify drugs which do not target actin but which are able to inhibit proteins involved in the initiation of actin filament elongation such as wiskostatin, an inhibitor of N-WASP (Peterson et al. Nat. Struct. Mol. Biol., 2004, 11, 747-755), CK666, an inhibitor of Arp2/3 (Nolen et al., Nature, 2009, 460, 1031-1034) and SMIFH2, an inhibitor of formins (Rivizi et al. Chem. Biol.,2009, 16, 1158-1168).

There is still a need of new drugs capable for impairing cytoskeleton dynamics, in particular actin dynamics in cell.

### SUMMARY OF THE INVENTION

The invention relates to a branched polyamine (BPA) having the general following formula (I):

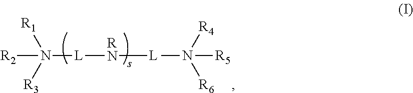

wherein:
Each wherein i is from 1 to 6, is independently selected from the group consisting of a hydrogen atom, and a group of formula (II)

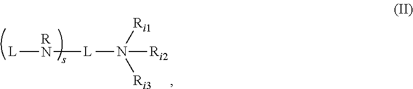

wherein:
Each Rᵢⱼ, wherein j is from 1 to 3, is independently selected from the group consisting of a hydrogen atom, and a group of formula (III):

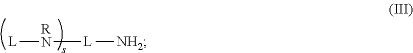

and
each of R₃, R₆ and Rᵢ₃ is independently present or not, if one of R₃, R₆ and Rᵢ₃ is present, the nitrogen atom bearing said group bears a positive charge;
Each L is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom such as N, O or S;
Each R is independently a hydrogen atom or a saturated hydrocarbon group comprising from 1 to 5 carbon atoms;
Each s is independently an integer from 0 to 5;
At least 3 groups among R₁, R₂, R₄ and R₅ are different from a hydrogen atom; and
Optionally at least one of N atom of the groups of formula (II) or (III), preferably at least one NH₂ group, is substituted with a label, preferably a fluorophore group and/or is in the form of an ammonium.

In some embodiments, said branched polyamine has one or several of the following features:
at least one NH₂ group of the groups of formula (II) or (III) is substituted with a fluorophore group, and/or
at least one L group comprises at least one oxyethylene —CH₂—CH₂—O— moiety, and/or
s is 0.

In some other embodiments, said branched polyamine is such that:
L group depicted in formula (I) is a hydrocarbon group, preferably a saturated hydrocarbon group, having from 4 to 10 carbon atoms, and/or
L groups depicted in formula (II) or (III) are a hydrocarbon group, preferably saturated hydrocarbon, group having from 2 to 6 carbon atoms.

In other embodiments, said branched polyamine is a compound of formula (Ia) formula (Ia) or a salt thereof:

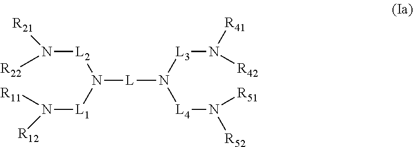

Wherein:
L is as described above in formula (I), preferably a saturated or unsaturated C₃-C₁₀ hydrocarbons group, preferably —(CH₂)_{d}— with d is an integer from 3 to 10,
L1, L2, L3, and L4 are independently selected among —(CH₂)ₑ— moieties with e an integer from 2 to 6,
R₁₁, R₁₂, R₂₁, R₂₂, R₄₁, R₄₂, R₅₁ and R₅₂ are independently selected from the group consisting of H, —Z, —(CH₂)_{f}—NH₂ and —(CH2)_{f}—NH—Z wherein Z is a label moiety, preferably a fluorophore, and f is an integer from 2 to 6.

In some embodiments, the branched polyamine comprises at least one fluorophore group. For instance said branched polyamine may be a compound of a compound of formula (Ic) or a salt thereof:

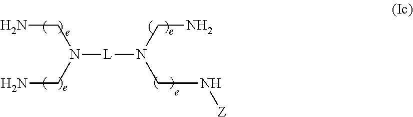

Wherein:
e is an integer from 2 to 6, preferably 3,
L is —(CH2)d- with d an integer from 3 to 10, preferably from 4 to 10 and more preferably from 7 to 10, and
Z is a fluorophore group selected among cyanine derivatives.

Particular branched polyamines of the invention are for instance:

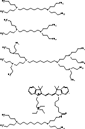 [SMILES: NCCCN(CCCN)CCCCCCCCN(CCCN)CCCN ; NCCCN(CCCN)CCCCCCN(CCCN)CCCN ; NCCCN(CCCN)CCCN(CCCCCCCCN(CCCN(CCN)CCN)CCCN(CCN)CCN)CCCN(CCN)CCN ; CC[N+](CC)(CC)CCC[N+]1=C(/C=C/C=C2/N(CCCCCC(=O)NCCCN(CCCN)CCCCCCCCN(CCCN)CCCN)c3ccccc3C2(C)C)C(C)(C)c2ccccc21]

and salts thereof.

In another aspect, the invention relates to a macrocyclic polyamine of formula (V):

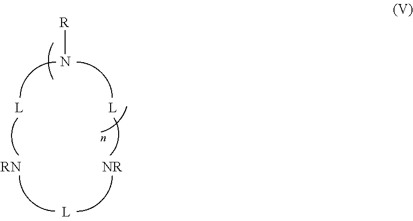

or a salt thereof, wherein
Each L group is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom such as N, O or S and
Each R is independently a hydrogen atom, a saturated hydrocarbon group comprising from 1 to 5 carbon atoms or a label moiety, preferably a fluorophore moiety. Preferably, each R group is a C₁-C₅ saturated hydrocarbon group or H; and
n is an integer from 1 to 33; and

preferably, said macrocyclic polyamine is different from anyone of the compounds of formula (VI)

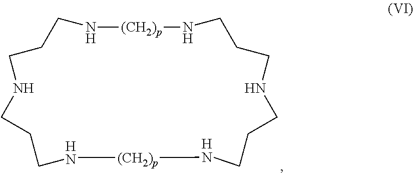

wherein p is 3, 7 or 10.

An additional object of the invention is a polyamine derivative comprising at least two polyamine moieties wherein:
the polyamine moieties independently derives from a branched polyamine or a macrocyclic polyamine as defined above and wherein:
   The at least two polyamine moieties being linked by linkers, wherein each linker is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom such as N, O or S, or
   the polyamine moieties further comprise a core unit to which the at least two polyamine moieties are linked by linkers, wherein each linker is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom such as N, O or S.

In some embodiments, the core unit of said polyamine derivatives comprises an aliphatic cycle or an aromatic cycle, preferably selected among a cycloalkane, a benzene or a naphthalene group.

In some other embodiments, the branched polyamine, the macrocyclic polyamine, or the polyamine derivative as defined herein comprises at least one nitrogen atom in the form of an ammonium. Accordingly, said compound may be used for complexing at least one organic or inorganic compound bearing at least one negative charge, wherein said organic or inorganic compound is preferably selected from biological compounds, such as proteins comprising at least one aspartic acid residue, phosphate site, and/or glutamic acid residue and biological compounds comprising at least one nucleotide. Preferably, said use is an in vitro use. The organic compound may be selected from actin protein, phospholipids and Tau protein.

A further object of the invention is the use, preferably in vitro, of a branched polyamine, a macrocyclic polyamine of formula (V) or a polyamine derivative as defined above as a cellular modulator of motility and/or for slowing down an actin-based process in a cell. The compounds of the invention may be used in vitro for promoting lamellipodia growth and/or for altering, preferably slowing-down, actin dynamics in a cell. In a general aspect, the compounds of the invention may be used as a pharmacological or biological research tool.

The invention also relates to the use of a branched polyamine, a macrocyclic polyamine or a polyamine derivative as defined herein, for use in the treatment of a disease involving protein-protein interaction, protein-nucleic acid interaction and/or nucleic acid-nucleic acid interaction, said disease preferably implying the cytoskeleton, cell migration, cell division, neurodegenerative diseases, such as Alzheimer's disease, diseases implying prions, gene transfer, through interaction with oligo(poly)nucleotide sequences, siRNAs, developmental biology, miRNAs, reparation of cell contacts and cancer.

In particular, the compounds of the invention may be used in the treatment of cancer, preferably the treatment of metastasis of cancer.

The invention further relates to a method of treating a disease involving biological compounds, preferably chosen among proteins comprising at least one aspartic acid residue, phosphate site and/or glutamic acid residue, and compounds comprising at least one nucleotide, comprising administering to a patient in need thereof a therapeutically effective amount of at least one branched polyamine, macrocyclic polyamine or polyamine derivative of the invention, optionally complexed with at least one organic or inorganic compound comprising at least one negative charge as defined above, under conditions effective to lead to a beneficial therapeutic effect.

The invention also relates to a kit comprising at least one compound selected among
a branched polyamine, a macrocyclic polyamine of formula (V) or (VI) and a polyamine derivative as defined herein, and
optionally monomeric actin and/or actin filaments.

Said kit may further comprise:
at least one additional compound selected in the group consisting of latrunculin A and B, cytochalasin D, jasplakinolide, witokostatin, CK666, SMIFH2, blebbistatin, ML-7, Y27632, ADF, Arp2/3, an actin nucleation agent such as ActA, IscA, RickA, WASp, N-WASP, pWa and SCAR-WAVE proteins, formins, spire, profilin, gelsolin, capping proteins, a cross-linking protein such as alpha-actinin, fascin, EF-1, Scruin, villin, dematin, fimbrin, spectrin, dystrophin, ABP 120, filamin, and one of their mixtures, and/or
a reagent such as a buffer, culture medium and the like, and/or
a cell or a cell culture and/or
a device such as microplates, a detection meas and the like, and/or
a detection compound, in particular a compound for the detection of actin structures such as a labeled phalloidin (e.g. alexa 488 phalloidin) or a labeled antibody directed against actin, and/or
written instructions.

Such a kit may be useful for implementing the in vitro methods on the in vitro use according to the invention.

The invention further relates to an in vitro method for studying assembly-disassembly dynamics of actin filaments or lamellipodium growth in a cell, comprising the steps of:
(a) providing a compound of the invention,
(b) contacting the compound of step (a) with a cell, and
(c) observing the assembly-disassembly dynamics of actin filaments or lamellipodium growth in said cell.

The invention further relates to the use of a compound according to the invention and comprising a label moiety, as an imaging tool for visualizing an actin structure, preferably in a cell.

### DESCRIPTION OF THE FIGURES

FIG. 1 shows the chemical structures of five synthetic polyamines. a) C7 cyclic superpolyamine (C₇N₆ MPA); b) C6 branched polyamine (C₆N₆ BPA); c) C8 branched polyamine, first generation (C₈N₆ BPA); d) C8 branched polyamine second generation (C₈N₁₄ BPA′); e) Fluorescent compound Cy3-C₈N₆ BPA. Cx designates the length of the central carbon chain(s) linking the amino subunits. All compounds were stored under inert atmosphere and used as their hydrochloride salts.

FIG. 2 shows that polyamine compounds promote growth of lamellipodia. Phase contrast images of NIH3T3 cells before and after incubation with (a) 100 μM cyclic C₈N₆ BPA and (b) with 100 μM C₇N₆ MPA. Zooms of selected zones are represented below each panel. Note that lamellipodia extend preferentially on growth fronts, but lamellipodia also grow on the cells sides. Neighboring cells establish contacts by bridging their lamellipodia (in a, region 2). Scale bars: 20 μm. (c) Live cells experiments were performed and the increase in the cells projected area was plotted for each compound and concentrations; here the Inventors show the 3D plots for C₈N₆ BPA. (d) 2D plot of the area increase as a function of time for C₈N₆ BPA. (Number of cells analyzed for each concentration: n(1.25 μM)=4; n(2.5 μM)=6; n(5 μM)=4 cells; n(10 μM)=10; n(25 μM)=14; n(50 μM)=16; n(100 μM)=9). Data is shown as mean±s.d.

FIG. 3 shows that the growth of lamellipodia is associated with the formation of focal contacts. (a) Control NIH3T3 cell stained for actin (green) and paxillin (red). Scale bar: 20 μm. (b) NIH3T3 cell 20 min after addition of 100 μM C₈N₆ BPA. (c) NIH3T3 cell 20 min after addition of 100 μM C₇N₆ MPA. White arrows in (b) and (c) show the lamellipodia protrusion after the addition of the polyamine compounds; arrowheads show also bundling of actin in (b). Highlighted regions are shown in more detail in the right panels. Scale bars 10 μm. (d-e) Growth occurs upon injection. NIH3T3 cell before injection (d); and lamellipodia growth 10 sec after injection of 300 μM C₇N₆ MPA. Scale bar: 5 μm; (f) The compounds enter the cells. Experiments with C₈N₆ BPA-Cy3 show that the polyamines enter the cells. Data is shown as mean±s.d. *P<0.001 (Student's t-test).

FIG. 4 shows that cyclic and branched polyamines induce ATP sensitive bundling of actin filaments. (a-d) MgATP-G-actin (4 μM, 10% pyrenyl-labeled) was polymerized by addition of 1 mM MgCl₂ and 0.1 M KCl in the absence and presence of C₇N₆ MPA (a, b) or C₈N₆ BPA (c, d) at the indicated concentrations and in the presence of ATP at 0.2 mM (a, b) or at the indicated concentrations (c, d). Spontaneous polymerization was monitored by the increase in intensity of light scattering at 310 nm (a-b) and by the simultaneous increase in pyrenyl-actin fluorescence, recorded on the same samples (c-d). (e-f) Electron microscopy observation of solutions of actin (2 μM) in physiological ionic strength (e) or low ionic strength (f) in the absence (left panel) or in the presence (right panel) of 300 μM C₇N₆ MPA.

FIG. 5(a) shows that C₇N₆ MPA promotes lamellipodia growth even when contractility is inhibited. (From top to bottom) NIH3T3 fibroblast before and after incubation 20 min with 100 μM C₇N₆ MPA; NIH3T3 cell after incubation 30 min with 30 μM Blebbistatin before and after 20 min with 100 μM C₇N₆ MPA; Cell incubated 30 min with 10 μM ML7 before and after 20 min with 100 μM C₇N₆ MPA; Cell incubated for 30 min with 10 μM Y27632 before and after 20 min with 100 μM C₇N₆ MPA. FIG. 5(b) shows the polyamines effect in the presence of Cytochalasin D and Latrunculin A. Actin specificity and barbed end target. (From left to right and top to bottom) 1 μM Cytochalasin D, 1.5 μM Latrunculin A, 1 μM Cytochalasin D followed by 100 μM C₇N₆ MPA, 1.5 μM Latrunculin A followed by 100 μM C₇N₆ MPA, 100 μM C₇N₆ MPA followed by 1 μM Cytochalasin D and 100 μM C₇N₆ MPA followed by 1.5 μM Latrunculin A. FIG. 5(c) shows C₇N₆ MPA effect in the presence of PIK3 kinase inhibitor LY294002. (Left) NIH3T3 cell after incubation 50 min with 50 μM LY294002. (Right) Cell incubated for 50 min with 50 μM LY294002 before and after 20 min with 100 μM C₇N₆ MPA. Scale bar: 20 μm.

FIG. 6 shows that cyclic polyamines slow down filament dynamics at barbed ends specifically, without affecting the thermodynamic stability of the filaments.

FIG. 6(Aa): Barbed end and pointed end growth were monitored using pyrenyl-actin fluorescence in the presence of 2.5 μM MgATP-G-actin. Rates are normalized taking as 1 the value measured in the absence of C₇N₆. FIG. 6(Ab): Binding of FH₁-FH₂ formin construct to barbed ends protects from the inhibition by C₇N₆ MPA. Conditions are as in (Aa). FIG. 6(Ac): Dilution-induced depolymerization at barbed ends was measured at the indicated concentrations of C₇N₆ MPA. FIG. 6(Ad): Increasing ionic strength weakens the inhibition of barbed end growth by C₇N₆ MPA. Barbed end growth rates were measured at the indicated concentrations of KCl in the absence (blue) and presence (red) of 100 μM C₇N₆ MPA. (Right) The ADF-induced increase in rate of depolymerization is greatly weakened by C₇N₆ MPA and C₈N₆ BPA. FIGS. 6(Ba-b): Time courses of ADF-induced rapid depolymerization of gelsolin-capped filaments in the absence (Ba) and presence (Bb) of 0.15 mM C₈N₆ BPA. FIG. 6(Bc): SDS-PAGE analysis of the pellets and supernatants of samples of F-actin (3 μM) incubated with ADF and with or without 0.3 mM C₇N₆ MPA or C₈N₆ BPA for one hour before being centrifuged at 400 000×g for 20 min. The steady state of actin assembly is not affected.

FIG. 7(a) shows that polyamines slow down the propulsive actin-based movement of N-WASP-coated beads in a reconstituted motility assay. (Left) Time-lapse phase contrast images of actin-based movement of N-WASP-coated beads in a reconstituted motility assay (see Methods). Scale bar: 15 μm. (Right) Measured bead velocity of sustained actin-based propulsion in the absence (open circles) and in the presence (closed circles) of 50 μM C₈N₆ BPA added two minutes after placing the beads in the medium (time zero). FIG. 7(b) shows that actin retrograde flow is slowed down in the presence of polyamines. (Left) Phase contrast images of the lamellopodia region of a REF 52 cell before and after the addition of C₈N₆ BPA. Scale bar: 5 μm. Kymographs of the highlighted region (dashed line) show a clear reduction in the actin retrograde flow. (Right) Graphic showing the measured retrograde flow velocity. (n=6 cells; N=6) Data is shown as mean±s.d. *P<0.05 (Students's t-test).

FIG. 8 shows that polyamines enhance nucleation by capping proteins. FIG. 8(a): Critical concentration plots with free barbed ends and in the presence of Capping Protein, with and without C₈N₆ BPA. FIG. 8(b): Effect of Capping Protein on F-actin assembled at steady state (1 μM total actin) in the absence and presence of 100 μM C₈N₆ BPA. Note that the experimental data points show a sigmoidal dependence on CP concentration due to the known non-linear relationship between the critical concentration and the extent of barbed end capping.

FIG. 9 shows that lamellipodia are extending upon addition of synthetic polyamines for different cells types. (a) USO2 Human Osteosarcoma cells; (b) SV80 cells; (c) REF52 cells. Cells are shown 20 minutes after addition of the drug. Scale bar: 20 μm. Inset images, scale bar: 5 μm.

FIG. 10 shows the efficiency of the four polyamine compounds. Live cells experiments were performed and the increase in cells projected area was plotted for each compound and concentrations (n=10). (a-c) Area increase for each compound. (d-g) Graphs comparing the area increase at the same concentration for three different compounds. Cell areas were larger than 940 μm² at time 0, when drugs were added. Data is shown as mean±s.d.

FIG. 11 shows that Polyamine C₇N₆-MPA reduces SW480 cell migration without evident effects on viability. (a) Using Boyden chamber migration assays, 100 μM treated SW480 cells showed a two-fold decrease in migration compared with that of control cells (*P=0.03, Student's t-test). (b) Cells were grown under normoxic conditions and viable cells were counted by trypan-blue exclusion method after 24 h (blue line) and 48 h (red line) exposure to increasing concentrations of polyamine C₇N₆ (1 nM-1 mM). No significant differences were observed between control and treated cells, even at the highest concentration (1 mM). Data is shown as mean±s.d.

FIG. 12 shows that synthetic polyamines promotion rapid growth of lamellipodia. (a-b) After cell rounding promoted by short incubation with trypsin, (c) cells spread within 20 minutes incubation of 100 μM C₇N₆ MPA. Projected areas were similar to control cells. (d-e) Promoted growth occurs in the absence of serum. (d) Cell in the absence of serum; (e) after 20 min of incubation with 100 μM C₇N₆ MPA a lamellipodia growth is observed. (Actin, in green; paxillin, in red). Scale bar: 20 μm.

FIG. 13 shows that polyamines enter the cells. (a) (Left) DIC and (Right) autofluorescence signal of NIH3T3 cells in the Cy3 channel. Note the absence of fluorescence signal compared to (b). (b) (Left) DIC image of lamellipodia growth after drug addition. (Right) Fluorescence image of cells after the addition of 100 μM Cy3-labeled C8N6 BPA for 20 min. The images show that the polyamines enter the cells. Note in particular the dark appearance of the nucleus confirming that indeed the compounds entered the cells. Lower images show a zoomed region of the highlighted areas. Scale bar 20 μm.

FIG. 14 shows that polyamine stabilizes actin filaments in Triton-treated cells like Jasplakinolide (a,b) Control and triton-treated NIH3T3 cells. (c,d) Cells after 10 min incubation of 1 μM Jasplakinolide (c) and after the addition of 0.1% Triton (d). (e,f) Cells after the addition of 100 μM C₈N₆ BPA (e) and after the addition of 0.1% Triton (f). Scale bar: 20 μm.

FIG. 15 shows that polyamines have no effect on microtubules and intermediate filaments organization. (a) (Left) Fluorescence microscopy images of microtubule organization for control NIH3T3 cells. (Right) Cells after 20 min incubation with 100 μM C₈N₆ BPA. Note that microtubules enter the lamellipodia. Lower panels show a magnification of cell lamellipodia showing no effect of polyamines in their organization. Scale bar: 20 μm. (b) (Top) Actin and intermediate filaments (IF) (vimentin) staining for control NIH3T3 fibroblasts. (Bottom) Cells after the addition of 100 μM C₈N₆ BPA. Note that intermediate filaments do not enter the lamellipodia. Right panels show the merge of the highlighted regions. Scale bar: 10 μm.

FIG. 16 shows that critical concentration does not change in the presence of MPA/BPA. Critical concentration plots with free barbed ends with and without (a) C₇N₆ MPA and (b) C₈N₆ BPA. The critical concentration plots were not significantly affected by the polyamines, except for a slight interference of the light scattering due to bundling, which caused a 10% increase in slope for C₇N₆ MPA.

FIG. 17 shows that ineffective triggering of actin polymerisation in vitro by Neomycin. As shown in (a), neomycin does not promote polymerization of actin in contrast to BPA in the same experimental conditions (G-buffer, LS: light scattering). In addition, elongation at the barbed end of spectrin seeds is not inhibited by neomycin (see (b)) in contrast to the inhibition with BPA (zoomed in the right side of panel (b)).

### DETAILED DESCRIPTION OF THE INVENTION

The instant invention is concerned with polyamines, in particular macrocyclic polyamines and branched acyclic polyamines, and derivatives thereof. Preferred compounds are thus depicted in formulae (A), (B), (I), (Ia), (Ib), (Ic), (Id), (V) and (Va) defined herebelow. As explained in the above section dedicated to the background of the invention, the set of drugs able to disrupt actin dynamics is quite limited and only encompasses drugs capable for inhibiting actin polymerization or for stabilizing actin filaments. For the first time, the Inventors showed that actin dynamics in cells was impaired by polyamines, in particular branched polyamines, through mechanisms which were dramatically distinct from those showed for cytoskeletal drugs described in the prior art.

More precisely, the Inventors showed that macrocyclic polyamines and branched polyamines display unique properties when contacting with cells. Indeed, as fully-illustrated in Example 2, the Inventors demonstrated that the compounds of the invention, preferably branched polyamines, entered the cells and induced specific growth of actin-enriched lamellipodia within few minutes in various cell lines. The Inventors further showed that the compounds of the invention specifically targeted actin and had no effect on other biological targets which may be involved in lamellipodia formation. Noteworthy, immunofluorescence assay suggested that the compounds of the invention neither impaired plasma membrane nor the organization of microtubules and intermediate filaments. The Inventors also demonstrated that the compounds of the invention did not inhibit PI3 kinase and did not require cell contractility to promote lamellipodia growth. By contrast, the compounds of the invention are unable to promote cell lamellipodia in the presence of actin polymerization inhibitors.

Finally, the Inventors further showed that the compounds of the invention were able to inhibit cell migration.

The analysis of the effects of the compounds on filament assembly dynamics and its regulation in cells indicated that the polyamines of the invention did not affect the stability of actin filaments but acted through two complementary ways, namely by slowing down filament dynamics and by enhancing actin nucleation. The polyamines of the invention, especially the branched polyamines of the invention, are thus unique tools for investigating actin cytoskeleton in motile and morphogenetic processes and may be used as therapeutic agents for preventing or treating disorders involving dysfunction of actin cytoskeleton, such as metastasis and proliferation.

Definitions

According to the present invention, a “synthetic polyamine” is a molecule with three or more potentially protonable nitrogen atoms that can coordinate to an anionic center through a set of supramolecular interactions.

According to the present invention, a “branched polyamine” is a molecule bearing a set of protonable nitrogen atoms attached to a branched backbone, namely a backbone having at least three arms.

According to the present invention, a “macrocycle” is, as defined by IUPAC, “a cyclic macromolecule or a macromolecular cyclic portion of a molecule”. May be considered as macrocycle, all molecules containing a ring of seven or more atoms. A macrocyclic polyamine can also be defined as a cyclic molecule with three or more potentially protonable nitrogen atoms that can coordinate to an anionic center.

According to the present invention, a “polyamine” is an organic compound having two or more primary, secondary or tertiary amino groups (—NH₂, —NH— or —NR— sites). This class of compounds includes spermidine H₂N—((CH₂)₄—NH—)₂—H, and spermine H₂N—((CH₂)₄—NH—)₃—H. A polyamine may also comprise one or several quaternary ammonium.

According to the invention, a “heteroatom” is an atom different from carbon and hydrogen atoms, such as nitrogen, oxygen and sulphur atoms.

According to the invention, the term “aromatic” refers to a monocyclic or polycyclic hydrocarbon aromatic group, optionally comprising at least one heteroatom as defined above, such as phenyl, naphthyl, anthracenyl, pyrrolyl, thiophenyl, furanyl, pyridinyl, pyrimidinyl, pyrazinyl, triazinyl, imidazolyl, thiazolyl, oxazolyl and indolyl groups.

According to the invention, a “neutral linker” is an organic radical such as for example an alkyl radical, which does not carry a positive or negative charge.

According to the invention, a “binding subunit” is a unit comprising receptor functions, which can bind to a substrate having complementary interaction properties. In particular, the binding units themselves must contain a suitable array of interaction sites capable of forming intermolecular bonds to the anionic sites of the substrate. Macrocycle or macropolycyclic polyammonium molecules, which contain binding units, may complex strongly and selectively anions. The complexation site consists of several positively charged binding sites arranged around a cavity defined by the macropolycyclic architecture. According to the invention, a “label group” or “a label moiety” is a chemical group appropriate for allowing detection of the compound of the invention and may be any chemical group that can be identified and/or quantified by any technique of analysis known in the art. Among labels for detection can be cited fluorescent probes, such as fluorescein, quantum dots, cyanine dyes Cy3 and Cy5, Alexa Fluor dyes, Dylight fluor dyes, IRIS Dyes, Seta dyes, SeTau dyes, SRfluor dyes, Square dyes or carboxytetramethylrhodamine (TAMRA); rhodamine, phycoerythrin, phycocyanin, allophycocyanin, o-phthaldehyde, and fluorescamine, a phosphorescent label, a chemiluminescent label or bioluminescent label such as luminal or isoluminol, Nuclear Magnetic Resonance (NMR) tags, such as xenon or lanthanides (in particular terbium Tb or europium Eu); magnetic resonance imaging (MRI) contrast agents such as Gd chelates; mass spectrometry tags such as tris(2,4,6-trimethoxyphenyl)phosphonium (TMPP) or isotope-coded tags; infrared (IR) tags; positron emission tomography (PET) tags; single-photon emission computed tomography (SPECT) tags; a radio-isotope such as tritium or deuterium atoms; microscopy tags such as gold nanoparticles.

As used herein, a fluorophore moiety, or fluorophore group, mainly refers to a chemical group that can re-emit light upon light excitation. In the context of the invention, this chemical moiety may be linked directly or through a spacer to the polyamine structure. Fluorophore moiety may derive from proteins and peptides, small organic compounds, synthetic oligomers and polymers, and multi-component systems and encompass:
Fluorescent proteins such as GFP (green), YFP (yellow) and RFP (red),
Xanthene derivatives such as fluorescein, rhodamine, Oregon green, eosin, and Texas red
Cyanine derivatives such as cyanine, indocarbocyanine, oxacarbocyanine, thiacarbocyanine, and merocyanine, e.g. Cy3 and Cy5 derivatives,
Naphthalene derivatives such as dansyl and prodan derivatives,
Coumarin derivatives,
oxadiazole derivatives such as pyridyloxazole, nitrobenzoxadiazole and benzoxadiazol,
Pyrene derivatives such as cascade blue etc.
Oxazine derivatives such as Nile red, Nile blue, cresyl violet, oxazine 170 etc.
Acridine derivatives such as proflavin, acridine orange, acridine yellow etc.
Arylmethine derivatives such as auramine, crystal violet, malachite green, and
Tetrapyrrole derivatives such as porphin, phthalocyanine, bilirubin.

Preferentially, the label is a fluorophore moiety, in particular a cyanine derivative as Cy-3 dye to form a Cy-3 dye labeled-polyamine.

According to the invention, the “core unit” is an organic radical comprising at least one aliphatic or aromatic ring.

Within the context of the present invention, the term “treatment” or “treating” includes the curative or preventive treatment, including for instance the retardation of the disease, asuppression or a reduction of the symptoms, an improvement in a subject condition or survival, or an increase in cognitive function.

As used herein, the verb “to comprise” is used in its non-limiting sense to mean that items following the word are included, but items not specifically mentioned are not excluded. In some embodiments, the verb may be interchangeably used for “consisting essentially of” or “consisting of”. In addition, reference to an element by the indefinite article “a” or “an” does not exclude the possibility that more than one of the element is present, unless the context clearly requires that there be one and only one of the elements. The indefinite article “a” or “an” thus usually means “at least one”. All patent and literature references cited in the present specification are hereby incorporated by reference in their entirety.

General Items of the Invention

In a general aspect, the invention relates to the following items 1 to 24:
1. A branched polyamine (BPA) having the general following formula (A):

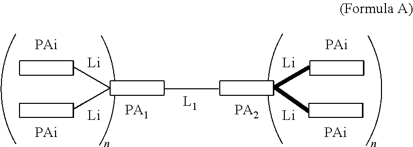

wherein
L1 and Li are neutral linkers with i being comprised from 3 to 5, said neutral linkers being identical or different hydrocarbon groups and chosen from the linear —(CH₂)p- polymethylene chains, wherein p is comprised from 2 to 18 carbon atoms, or branched saturated chains, or may contain unsaturated units, such as double bonds, triple bonds or aromatic units, as well as heteroatomic chains, such as oxyethylene units —CH₂—CH₂—O—CH₂—CH₂— and the like,
PA1, PA2 and PAi are polyamine binding subunits with i being comprised from 3 to 5 binding subunits being chosen among the groups

NR—(CH₂)q-NR—[(CH₂)r-NR]s-
wherein s is comprised from 0 to 5, advantageously from 0 to 3, q and r are identical or different and comprised from 2 and 5, and R is an H atom or is chosen among the alkyl groups, and
n, defining a number of units comprising the association of the neutral linker (Li) and the polyamine binding subunit (PAi), is advantageously equal to 1, 2 and 3, but not limited to this range,

except the synthetic polyamines for which:
(i) L1 has p equal to 3, 7, 9 or 10,
(ii) PA1 and PA2 are identical, s is equal to 1, R is an H atom, and for which:
   q and r are identical and each one equal to 2, when p is equal to 9, or
   q and r are identical and each one equal to 3, when p is equal to 3, 7 or 10, and
(iii) n is equal to 0.

2. A macrocyclic polyamine (MPA) having the following general formula (B):

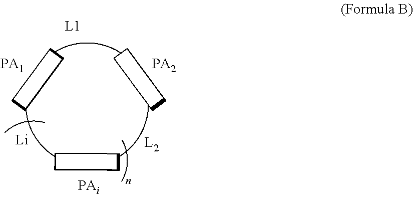

wherein
L1, L2 and Li are neutral linkers with i being comprised from 3 to 5, said neutral linkers being identical or different hydrocarbon groups and chosen from the linear —(CH₂)p- polymethylene chains, wherein p is comprised from 2 to 18 carbon atoms, or branched saturated chains, or may contain unsaturated units, such as double bonds, triple bonds or aromatic units, as well as heteroatomic chains, such as oxyethylene units —CH₂—CH₂—O—CH₂—CH₂— and the like,
PA1, PA2 and PAi are polyamine binding subunits with i being comprised from 3 to 5 binding subunits being chosen among the groups

NR—(CH₂)q-NR—[(CH₂)r-NR]s-
wherein s is comprised from 0 to 5, advantageously from 0 to 3, q and r are identical or different and comprised from 2 and 5, and R is an H atom or is chosen among the alkyl groups, and
n, defining a number of units comprising the association of the neutral linker (Li) and the polyamine binding subunit (PAi), is advantageously equal to 1, 2 and 3, but not limited to this range.

except the synthetic polyamines for which:
(i) L1 and L2 are identical, p being equal to 3, 7, 9 or 10,
(ii) PA1 and PA2 are identical, s is equal to 1, R is an H atom, and for which:
   q and r are identical and each one equal to 2, when p is equal to 9, or
   q and r are identical and each one equal to 3, when p is equal to 3, 7 or 10, and
(iii) n is equal to 0.

3. Synthetic polyamine according to item 1 or 2, wherein n is equal to 0.

4. Synthetic polyamine according to item 3, wherein PA1 and PA2 are identical.

5. Synthetic polyamine according to item 3, wherein PA1 and PA2 are different.

6. Synthetic polyamine according to item 4 or 5, wherein L1 and L2 are identical.

7. Synthetic polyamine according to item 3 or 4, wherein L1 and L2 are different.

8. Synthetic polyamine according to any one of items 1 to 7, wherein n is equal to 1, comprising:
neutral linkers L1, L2 and L3, which are identical and polyamine binding subunits PA1, PA2 and PA3, which are identical or different; or
neutral linkers L1, L2 and L3, which are different and polyamine binding subunits PA1, PA2 and PA3, which are identical or different.

9. A Structure comprising at least one synthetic polyamine according to item 1 or 2, wherein
L1, L2 and Li are neutral linkers with i being comprised from 3 to 5, said neutral linkers being identical or different hydrocarbon groups and chosen among the linear —(CH₂)p- polymethylene chains, wherein p is comprised from 2 to 18 carbon atoms, or branched saturated chains, or may contain unsaturated units, such as double bonds, triple bonds or aromatic units, as well as heteroatomic chains, such as oxyethylene units —CH₂—CH₂—O—CH₂—CH₂— and the like.
PA1, PA2 and PAi are polyamine binding subunits with i being comprised from 3 to 5 binding subunits being chosen among the groups

—NR—(CH₂)_{q}—NR—[(CH₂)ᵣ—NR]ₛ—
wherein s is comprised from 0 to 5, advantageously from 0 to 3, q and r are identical or different and comprised from 2 and 5, and R is an H atom or is chosen among the alkyl groups, and wherein n, defining a number of units comprising the association of the neutral linker (Li) and the polyamine binding subunit (PAi), is comprised between 0 and 5, advantageously said synthetic polyamine is chosen from the synthetic polyamines for which:
(i) L1 and L2 are identical, p being equal to 3, 7, 9 or 10,
(ii) PA1 and PA2 are identical, s is equal to 1, R is an H atom, and for which:
   q and r are identical and each one equal to 2, when p is equal to 9, or
   q and r are identical and each one equal to 3, when p is equal to 3, 7 or 10, and
(iii) n is equal to 0,

said synthetic polyamine being linked with a neutral linker chosen among the linear —(CH₂)t- polymethylene chains, wherein t is comprised from 2 to 18 carbon atoms, or branched saturated chains, or may contain unsaturated units, such as double bonds, triple bonds or aromatic units, as well as heteroatomic chains, such as oxyethylene units —CH₂—CH₂—O—CH₂—CH₂— and the like.

10. Structure according to item 9, comprising besides at least one other synthetic polyamine, said at least one synthetic polyamine and said other synthetic polyamine (i) being identical or different, (ii) being of formula (A) as defined in item 8, and being linked each other via said neutral linker

11. Structure according to item 9, comprising at least two synthetic polyamines, identical or different, said synthetic polyamines being linked to a core unit by a said neutral linker.

12. Structure according to item 11, wherein the core unit is chosen among aliphatic or aromatic rings such as derived from cycloalkane or benzene, naphthalene or similar groups.

13. Structure according to any one of items 11 or 12, comprising at least three synthetic polyamines, identical or different, each said synthetic polyamine being linked with a said neutral linker, and all said neutral linkers are linked with the core unit and positioned all around said core unit forming a central unit.

14. Structure according to any one of items 11 or 12, comprising at least three synthetic polyamines, identical or different, each said synthetic polyamine being linked with a said neutral linker, and all said neutral linkers are linked with the core unit and positioned along said core unit forming a linear structure or an axis.

15. Structure according to item 9, wherein said at least one neutral linker is linked with at least another neutral linker or at least one polyamine binding subunit, said neutral linker and polyamine binding unit being defined in item 8.

16. Structure according to anyone of items 1 to 15 wherein amine functions are full or partially protonated in order to form a polyammonium cations structure.

17. Structure according to item 15, wherein at least one organic or inorganic anion molecule comprising at least one anion function, said anion function being preferably a carboxylate function, is complexed with said polyammonium cations structure.

18. Structure according to item 17, wherein said organic or inorganic anion is chosen among biological compounds, preferably chosen (i) among proteins comprising at least one aspartic acid residue, phosphate site and/or glutamic residue, and/or (ii) among molecules comprising at least one nucleotide and/or oligonucleotide.

19. Structure according to item 17, wherein said organic anion molecule comprising at least one anion function is chosen among actin protein, phospholipids and Tau protein.

20. Structure according to any one of the items 9 to 19 for use in the treatment of disease involving protein-protein interaction, protein-nucleic acid interaction and/or nucleic acid-nucleic acid interaction.

21. Structure according to item 20, wherein disease is chosen among diseases implying the cytoskeleton, cell migration, cell division, Alzheimer, diseases implying prions, gene transfer, through interaction with oligo(poly)nucleotide sequences, siRNAs, developmental biology, miRNAs, reparation of cell contacts and cancer.

22. Use of the synthetic polyamine according to items 1 to 8 and structure according to any one of items 9 to 19 in biological processes involving protein-protein interaction, protein-nucleic acid interaction and/or nucleic acid-nucleic acid interaction.

23. Method of preventing or treating a disease involving biological compounds, preferably chosen (i) among proteins comprising at least one aspartic acid residue, phosphate site and/or glutamic acid residue, and/or among (ii) molecules comprising at least one nucleotide and/or oligonucleotide, comprising administering to a patient in need thereof a therapeutically effective amount of the structure as defined in any one of items 16 to 19 under conditions effective to lead to a beneficial therapeutic effect.

24. A process of making a structure according to item 16, by putting the structure as defined in any one of items 1 to 15 into a weak acids medium in order to protonate some or all the polyamine functions.

Compounds of the Invention

In a more specific aspect, the invention relates to a branched polyamine of formula (I)

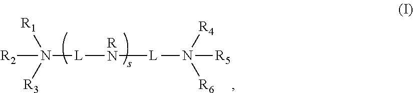

wherein:
Each wherein i is from 1 to 6, is independently selected from the group consisting of a hydrogen atom, and a group of formula (II)

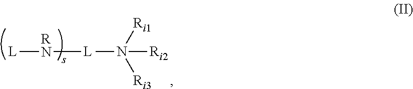

wherein:
Each Rᵢⱼ, wherein j is from 1 to 3, is independently selected from the group consisting of a hydrogen atom, and a group of formula (III):

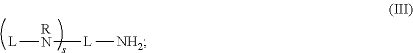

and each of R₃, R₆ and Rᵢ₃ is independently present or not, if one of R₃, R₆ and Rᵢ₃ is present, the nitrogen atom bearing said group bears a positive charge;
Each L is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom such as N, O or S;
Each R is independently a hydrogen atom or a saturated hydrocarbon group comprising from 1 to 5 carbon atoms;
Each s is independently an integer from 0 to 5;
At least 3 groups among R₁, R₂, R₄ and R₅ are different from a hydrogen atom; and
Optionally at least one of N atom of the groups of formula (II) or (III), preferably a NH₂ group, is substituted with a label, preferably a fluorophore group and/or is in the form of an ammonium.

The letter i of Rij group in formula (II) refers to the letter i of Ri group in formula (I). It goes without saying that if Ri is absent, Rij groups are also absent.

It is apparent that a compound of formula (I) may comprise several groups L and R. L groups are selected independently to each other. This means that the L groups present in a given compound may be different or identical, with proviso each L group present in the compound is a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom such as N, O or S

Similarly, R groups are selected independently to each other. A compound of formula (I) may also comprise in its specific formula several integers s which are independently selected to each other.

In some embodiments, each s present in formula (I), (II) and (III) is 0.

As used herein, a hydrocarbon group comprising from 2 to 18 carbon atoms encompasses C₂, C₃, C₄, C₅, C₆, C₇, C₈, C₉, C₁₀, C₁₁, C₁₂, C₁₃, C₁₄, C₁₅, C₁₆, C₁₇ and C₁₈ hydrocarbon groups.

In some embodiments, the L groups specified in formula (I) are selected among C₃-C₁₀, preferably C₄-C₁₀, unsaturated or saturated hydrocarbon groups and the L moieties specified in formula (II) and/or in formula (III) are selected among C₂-C₆ hydrocarbon groups, preferably C₂-C₆ saturated hydrocarbon groups.

As used herein, a hydrocarbon group interrupted by at least one heteroatom refers to a linear or branched, saturated or unsaturated, hydrocarbon group comprises one or several hydrocarbon subgroups linked together by one or several heteroatoms. More precisely, a hydrocarbon group interrupted by at least one heteroatom comprises one or several -A-X—B— moieties wherein (i) A and B are selected from linear or branched, saturated or unsaturated, hydrocarbon chains, or A and B form together a ring, and (ii) X is a heteroatom preferably selected from N, O and S.

In some embodiments, the compound of formula (I) comprises at least one L group comprising at least one oxyethylene moiety —CH₂—CH₂—O—.

In some other embodiments, the compound of formula (I) comprises at least one L group selected among —(CH₂)ₐ—(O—CH₂—CH₂)_{b}—(CH₂)_{c}— wherein a is an integer from 1 to 17, c is an integer from 1 to 17 and b is an integer from 0 to 8 with the proviso that 2≦a+2b+c≦18.

As used herein, an unsaturated hydrocarbon group refers to a hydrocarbon group comprising at least one double or triple bond. In some embodiments, at least one L group as specified in formula (I) comprises 1, 2, or 3 double and/or triple bonds.

As used herein, a saturated hydrocarbon group comprising from 1 to 5 carbon atoms encompass alkyl groups and cycloalkyl groups. C₁₋₅ cycloalkyl groups encompass cyclopropyl, methylcyclopropyl, cyclobutyl, methylcyclobutyl and cyclopentyl. C₁₋₅ alkyl groups encompass methyl, ethyl, propyl, isopropyl, isobutyl, tert-butyl, and pentyl.

As defined above, the polyamine of formula (I) may be uncharged or charged, namely bearing one or several positive charges depending on the presence or the absence of groups R₃, R₆ and Rᵢ₃. If all groups R₃, R₆ and Rᵢ₃ are absent, the compound may be uncharged if all the nitrogen atoms are trivalent. If at least one group among R₃, R₆ and Rᵢ₃ is present, at least one nitrogen atom of the compound is tetravalent and thus bears a positive charge.

If R₃, R₆ and Rᵢ₃ are absent, the compound may also bear positive charges if at least one nitrogen atom is protonated, for instance if at least one —NH₂ group present in R₁, R₂, R₃ or R₄ groups is in the form of an ammonium group —NH₃⁺.

When bearing one or several positive charges, the polyamine of formula (I) is associated with one or several counter-anions The counter-anion(s) Q⁻ may be selected among inorganic anions such as F⁻, Cl⁻, Br⁻, CO₃²⁻, PO₄³⁻, SO₄²⁻, NO₃²⁻ and the like.

The counter anion(s) may be also selected among organic molecules, preferably bearing a carboxylate or a phosphate group, such as acetate, succinate, lactate, citrate, maleate, tartrate, amino acids preferably glutamate and asparate, EDTA, nucleotides and the like.

The counter anion(s) may be also a macromolecule such as an oligonucleotide e.g. a DNA, a RNA, in particular a siRNA or a miRNA, a phospholipid or a protein comprising several aspartate or glutamate residues such as actin.

In some embodiments, the branched polyamine of formula (I) is such that:
R₃ and R₆ groups are absent and
R₁₃, R₂₃, R₄₃ and R₅₃ groups are also absent.

In other embodiments, the branched polyamine of formula (I) is such that:
R₃ and R₆ groups are present and are H and
R₁₃, R₂₃, R₄₃ and R₅₃ groups are present and are H.

In some other embodiments, the branched polyamine of formula (I) comprises one or several (1, 2, 3, 4 or 5) of the following features:
R₃, R₆ are, independently to each other, H or absent, and/or
R₁, R₂, R₄ and R₅ are of formula (II) wherein R₁₃, R₂₃, R₄₃ and R₅₃ groups, independently to each other H or absent, and/or
s integers are independently selected from 0, 1, 2 or 3, and/or
When s is not 0 in formula (II) and/or (III), the corresponding R groups are independently to each other H or a C₁-C₅ alkyl, and/or
L moieties are independently selected among saturated or unsaturated, linear C₂-C₁₈ hydrocarbon groups optionally substituted by one or more C₁-C₃ alkyl radicals and/or comprising at least one —(CH₂)₂—O— unit.

In some alternate or additional embodiments, the branched polyamine of formula (I) is such that:
each s present as specified in formula (I), (II) and/or (III) is 0 and/or
each L in formula (I) is independently selected among C₄-C₁₀, preferably C₃-C₁₀, saturated or unsaturated hydrocarbon groups and each L of formula (II) or (III) is a C₂-C₆, preferably saturated, hydrocarbon groups.

In some other alternate or additional embodiments, the branched polyamine of formula (I) is such that:
each s present as specified in formula (I), (II) and/or (III) is 0 and/or
each L in formula (I) is independently selected among C₄-C₁₀, preferably C₃-C₁₀, more preferably a C₇-C₁₀ such as a C₈, saturated hydrocarbon groups and each L of formula (II) or (III) is a C₂-C₆ such as a C₃, preferably saturated, hydrocarbon groups.

In some further embodiment, the invention relates to a branched polyamine compound of formula (I) wherein:
s as specified in formula (I) is 0,
R₃ and R₆ are absent, and
R₁, R₂, R₄ and R₅ are groups of formula (II) wherein s is 0, all Rᵢ₃ groups are absent and all Rᵢ₁ and Rᵢ₂ groups are independently selected among H and a moiety of formula (III) in which s is 0,

wherein optionally at least one of N atoms present in the groups of formula (II) or (III), preferably at least one of NH₂ groups, is further substituted with a label, preferably a fluorophore group and/or is in the form of an ammonium group.

Preferably, Z is a fluorophore moiety, for instance a cyanine derivative such as Cy3 dye.

In a more specific aspect, the invention relates to a branched polyamine of formula (Ia) or a salt thereof:

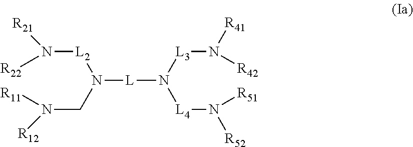

Wherein:
L is as described above in formula (I), preferably a saturated or unsaturated C₃-C₁₀ hydrocarbon group and more preferably —(CH₂)_{d}— with d is an integer from 3 to 10, preferably from 4 to 10, more preferably from 7 to 10 such as 8.
L₁, L₂, L₃, and L₄ are selected among L groups as defined in formula (I), and are preferably independently selected among —(CH₂)ₑ— with e an integer from 2 to 6,
R₁₁, R₁₂, R₂₁, R₂₂, R₄₁, R₄₂, R₅₁ and R₅₂ are independently selected to each other, from:
   H,
   a group of formula (III) as defined above in formula (I), preferably wherein s=0 and L is —(CH₂)_{f} with fan integer from 2 to 6, and wherein the N atom is optionally substituted with a label moiety, preferably a fluorophore, and
   a label moiety, preferably a fluorophore group.

An integer from 2 to 6, such as e as defined above, encompasses 2, 3, 4, 5 and 6.

An integer from 3 to 10, such as d as defined above, encompasses 3, 4, 5, 6, 7, 8, 9 and 10.

In preferred embodiments, L₁-L₄ moieties are identical. Alternatively or additionally, L groups potentially present in R₁₁, R₁₂, R₂₁, R₂₂, R₄₁, R₄₂, R₅₁ and R₅₂ are also identical.

In some alternate or additional embodiments, R₁₁, R₁₂, R₂₁, R₂₂, R₄₁, R₄₂, R₅₁ and R₅₂ are independently selected from the group consisting of
—H,
a label moiety —Z,
—(CH₂)_{f}—NH₂ and
—(CH₂)_{f}—NH—Z
wherein Z is a label moiety and f is an integer from 2 to 6.

Preferably, Z is a fluorophore moiety. Said fluorophore moiety may be selected among xanthene derivatives, cyanine derivatives, naphthalene derivatives, coumarin derivatives, oxadiazole derivatives, pyrene derivatives, oxazine derivatives, acridine derivatives, arylmethine derivatives, and tetrapyrrole derivatives. Z moiety may comprise a spacer, which links the fluorophore entity to the polyamine core. In some embodiments, Z moiety has a molecular weight which is lower than 5 000 g·mol⁻¹.

In other embodiments, Z moiety is a cyanine derivative such as Cy2, Cy3, Cy3B, CY3.5, Cy5, Cy5.5 and Cy7 dyes.

In a further aspect, the invention relates to a branched polyamine of formula (Ib) or a salt thereof:

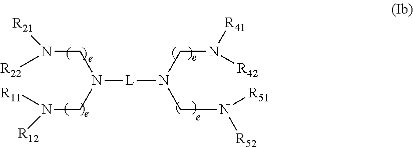

Wherein:
e is an integer from 2 to 6,
L is as defined above in formula (I), preferably —(CH₂)_{d}— with d an integer from 3 to 10, preferably from 4 to 10 and more preferably from 7 to 10 such as 8,
R₁₁, R₁₂, R₂₁, R₂₂, R₄₁, R₄₂, and R₅₁ are selected as follows:
   R₁₁, R₁₂, R₂₁, R₂₂, R₄₁, R₄₂, R₅₁ and R₅₂ groups are identical and selected from H and —(CH₂)_{f}—NH₂ with fan integer from 2 to 6, or
   (i) R₁₁, R₁₂, R₂₁, R₂₂, R₄₁, R₄₂ and R₅₁ groups are identical and selected from H and —(CH₂)_{f}—NH₂ with f an integer from 2 to 6, and (ii) R₅₂ is selected from Z and —(CH₂)_{f}—NH—Z, wherein Z is a label moiety, preferably a fluorophore moiety as defined above and f is an integer from 2 to 6.

In some additional embodiments, the invention relates to a branched polyamine of formula (Ic) or a salt thereof:

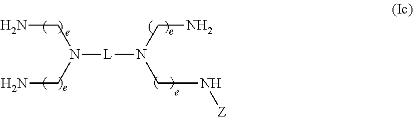

Wherein:
e is an integer from 2 to 6, preferably 3,
L is —(CH₂)_{d}— with d an integer from 3 to 10, preferably from 4 to 10 and more preferably from 7 to 10 such as 8, and
Z is a label moiety, preferably a fluorophore moiety, as defined herein.

Preferably, Z is selected among xanthene derivatives, cyanine derivatives, naphthalene derivatives, coumarin derivatives, oxadiazole derivatives, pyrene derivatives, oxazine derivatives, acridine derivatives, arylmethine derivatives, and tetrapyrrole derivatives. In some embodiments, Z moiety has a molecular weight which is lower than 5 000 g·mol⁻¹.

For instance, Z may be a cyanine derivative such as Cy2, Cy3, Cy3B, CY3.5, Cy5, Cy5.5 and Cy7 dyes.

In some other embodiments, the invention relates to a branched polyamine of formula (Id) or a salt thereof:

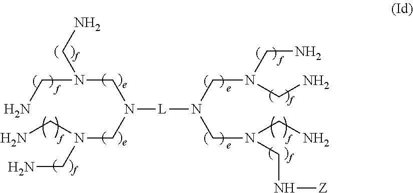

Wherein:
e is an integer from 2 to 6, preferably 3,
L is —(CH₂)_{d}— with d an integer from 3 to 10, preferably from 4 to 10 and more preferably from 7 to 10 such as 8, and
Z is a label moiety, preferably a fluorophore moiety as defined herein

Preferably, Z is a fluorophore moiety. Said fluorophore moiety may be selected among xanthene derivatives, cyanine derivatives, naphthalene derivatives, coumarin derivatives, oxadiazole derivatives, pyrene derivatives, oxazine derivatives, acridine derivatives, arylmethine derivatives, and tetrapyrrole derivatives. Z moiety may comprise a spacer, which links the fluorophore entity to the polyamine core. In some embodiments, Z moiety has a molecular weight which is lower than 5 000 g·mol−1.

For instance, Z may be a cyanine derivative such as Cy2, Cy3, Cy3B, CY3.5, Cy5, Cy5.5 and Cy7 dyes.

Examples of branched polyamines according to the invention are as follows:
N,N,N,N-[tetrakis(aminopropyl)trimethylenediamine (C₃N₆BPA):

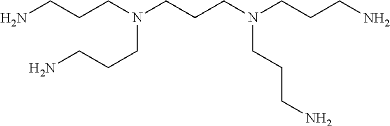 [SMILES: NCCCN(CCCN)CCCN(CCCN)CCCN]

N,N,N,′N′-tetrakis(aminopropyl)decamethylenediamine (C₁₀N₆BPA):

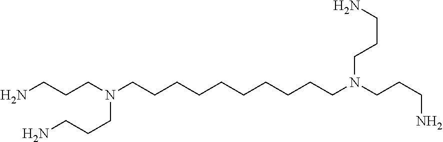 [SMILES: NCCCN(CCCN)CCCCCCCCCCN(CCCN)CCCN]

N,N,N′,N′-[tetrakis(aminopropyl)nonamethylenediamine (C₉N₆ BPA):

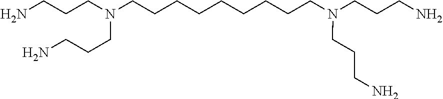 [SMILES: NCCCN(CCCN)CCCCCCCCCN(CCCN)CCCN]

N,N,N′,N′-[tetrakis(aminopropyl)octamethylenediamine (C₈N₆ BPA):

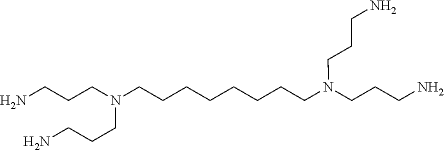 [SMILES: NCCCN(CCCN)CCCCCCCCN(CCCN)CCCN]

N,N,N′,N′-[tetrakis(aminopropyl)heptamethylenediamine (C₇N₆ BPA):

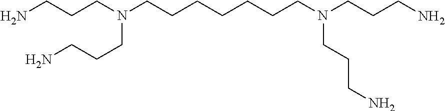 [SMILES: NCCCN(CCCN)CCCCCCCN(CCCN)CCCN]

N,N,N′,N′-[tetrakis(aminopropyl)hexamethylenediamine (C₆N₆ BPA):

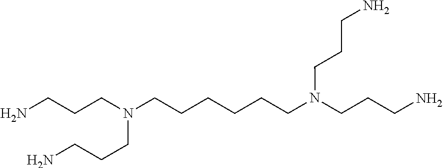 [SMILES: NCCCN(CCCN)CCCCCCN(CCCN)CCCN]

C₈N₁₄BPA

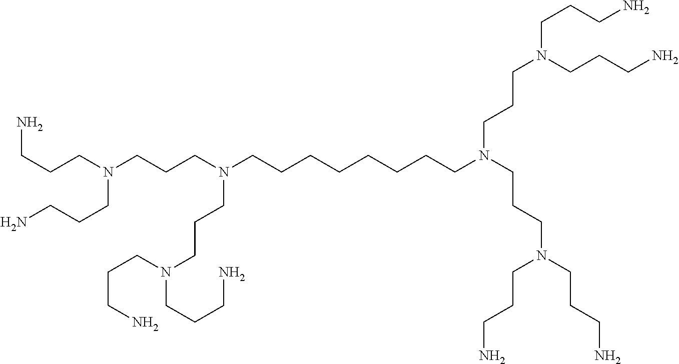 [SMILES: NCCCN(CCCN)CCCN(CCCCCCCCN(CCCN(CCCN)CCCN)CCCN(CCCN)CCCN)CCCN(CCCN)CCCN]

Cy-3 dye labeled-N,N,N′,N′-[tetrakis(aminopropyl)octamethylenediamine (C₈N₆ BPA-Cy3):

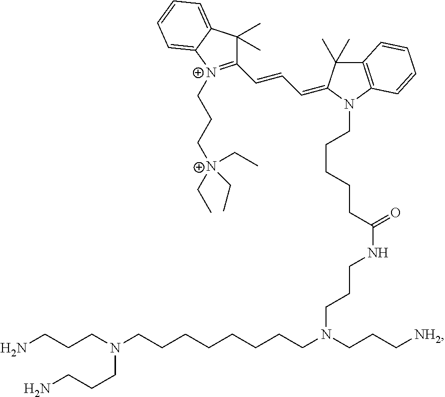 [SMILES: CC[N+](CC)(CC)CCC[N+]1=C(/C=C/C=C2/N(CCCCCC(=O)NCCCN(CCCN)CCCCCCCCN(CCCN)CCCN)c3ccccc3C2(C)C)C(C)(C)c2ccccc21]

2 Q⁻ wherein Q⁻ is a counter anion.

The invention also relates to salts of the above compounds, for instance hydrochloride thereof.

In an additional aspect, the invention relates to a macrocyclic polyamine of formula (V):

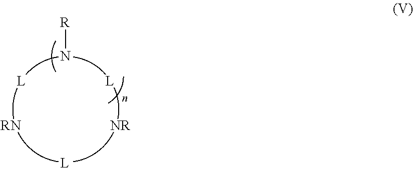

or a salt thereof, wherein
Each L group is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom such as N, O or S and
Each R is independently a hydrogen atom, a saturated hydrocarbon group comprising from 1 to 5 carbon atoms or a label moiety, preferably a fluorophore moiety as defined above for branched polyamine. Preferably, each R group is a C₁-C₅ saturated hydrocarbon group or H; and
n is an integer from 1 to 33;

In some embodiments, the macrocyclic polyamine of the invention is not a compound of formula (VI):

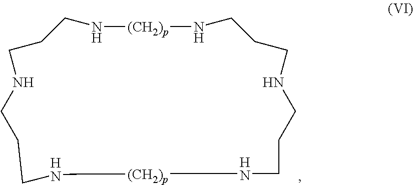

wherein p is 3, 7 or 10.

Preferably, the macrocyclic polyamine of formula (V) has one or several of the following features:
n is an integer from 1 to 20, preferably from 1 to 10, and more preferably from 1 to 5, for instance 4, and/or
each R is independently selected among C₁-C₅ saturated hydrocarbon group and H.

In some specific embodiments, the macrocyclic polyamine is selected among compounds of formula (Va) or a salt thereof:

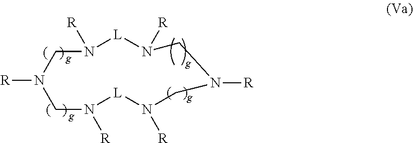

wherein:
each L group is as defined in formula (V). Preferably, L groups are independently selected among saturated or unsaturated C₃-C₁₀ hydrocarbon groups and more preferably, L groups are identical and are —(CH₂)ₚ— with p is an integer from 3 to 10, preferably from 4 to 10,
g is an integer selected among 2 to 6, and
R groups are as defined above in formula (V). Preferably, R groups are H.

In some embodiments, the macrocyclic compound of the invention is not a compound of formula (Va) wherein g is 3, L is —(CH₂)ₚ— with p=3, 7 or 10 and all R groups are H.

As in the case of branched polyamines as defined herein, the macrocyclic polyamines of the invention may bear one or several positive charges and thus may be associated with at least one counter-anion Q⁻ as defined above.

In a more general aspect, macrocyclic polyamines and branched polyamines as described herein may be partially or fully protonated, whereby said compounds are in the form of a polyammonium structure.

In a further aspect, the invention relates to a polyamine derivative. As used herein, a polyamine derivatives refers to a compound which comprises at least one polyamine derivative as defined in anyone of formulae (I) and (V) and in particular as defined in anyone of formulae (Ia), (Ib), (Ic), (Id) and (Va).

Preferably, the polyamine derivative of the invention comprises at least two polyamine moieties which are independently selected among branched polyamine moieties as defined in formula (I) and macrocyclic polyamines moieties as defined in formula (V).

At least two polyamine moieties encompass 2, 3, 4, 5, 6, 7, 8 or 10 polyamine moieties.

In some preferred embodiments, the polyamine derivatives comprise 2 or 3 polyamine moieties.

In some embodiments, the polyamine derivative of the invention comprises:
at least one polyamine moiety as defined in anyone of formulae (I), (Ia), (Ib), (Ic) or (Id) and at least one polyamine moiety as defined in anyone of formula (V) and (Va), or
at least two polyamine moieties as defined in anyone of formulae (I), (Ia), (Ib), (Ic) or (Id), or
at least two polyamine moieties as defined in anyone of formulae (V) and (Va)

The polyamine moieties present within the polyamine derivative may be identical or distinct. Moreover, the polyamine moieties may be uncharged or may bear one or several positive charges. In particular, the polyamine moieties may be fully or partially protonated and thus may be present in the form of polyammonium structures.

The polyamine moieties of said derivative may be linked together by one or several linkers. The linker(s) may be of any type. Preferably, the linkers are neutral, this means that the linkers are not charged. Typically, the linkers are independently selected among linear or branched, saturated or unsaturated, hydrocarbon groups comprising from 2 to 18 carbon atoms, optionally interrupted by at least one aromatic unit and/or at least one heteroatom such as O or S.

The chemical bond between a linker and a polyamine moiety may be of any type. For instance, a polyamine moiety may be linked to the linker through an amide bond or through a carbon-nitrogen bond.

In some other embodiments, the polyamine derivative comprises a core unit. As used herein, a core unit refers to an organic radical comprising at least one aliphatic or aromatic ring. Generally specific, the core unit may comprise from 5 to 100, preferably from 6 to 50, carbon atoms and optionally, one or several heteroatoms and/or one or several substituents such as alkyl groups, halogens, amino, hydroxyl, cyano, and the like. In some preferred embodiments, the core unit derives from a cycloalkane, benzene or naphthalene group.

The polyamine derivatives may be linked directly or through linkers to the core unit. In some embodiments, the polyamine derivative of the invention comprises at least two polyamine moieties, wherein the polyamine moieties are linked to the core unit by linkers, wherein each linker is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom such as N, O or S. In some embodiments, the linkers between the polyamine moieties and the core unit are neutral.

In a specific aspect, the invention relates to a branched polyamine, a macrocyclic polyamine or a polyamine derivative as defined above in the form of an ammonium salt. This means that at least one nitrogen atom present within said compounds bear a positive charged.

In some additional or alternative aspect, the amine functions of said branched polyamine, said macrocyclic polyamine or said polyamine derivative are full or partially protonated whereby said branched polyamine, said macrocyclic polyamine or said polyamine derivative is in the form of a polyammonium. The partial or the full protonation of amino groups may be obtained by contacting the compound of the invention with a medium comprising at least one weak acid.

In an additional aspect, the invention relates to a supramolecular complex comprising:
a compound selected among a branched polyamine of the invention, a macrocyclic polyamine of the invention and a polyamine derivative of the invention, and
a molecule having at least one carboxylate group and/or at least one phosphate group.

The molecule having at least one carboxylate group and/or at least one phosphate group may be selected among phospho lipids, proteins and peptides, in particular proteins and peptides comprising multiple aspartic and/or glutamic residue, nucleotides and oligonucleotides. Proteins of interest are, among others, actin and Tau proteins.

In a preferred embodiment, said protein of interest is actin.

In some other or additional embodiments, the supramolecular complex comprises a branched polyamine of the invention.

In a further aspect, the invention relates to the use of a compound of the invention, namely branched polyamine of the invention, a macrocyclic polyamine of the invention and a polyamine derivative of the invention for complexing a molecule having at least one carboxylate group and/or at least one phosphate group as defined hereabove. Preferably said use is an ex vivo use, in particular an in vitro use. In a further preferred embodiments, the compound of the invention is a branched polyamine.

Compositions, Kits, Methods and Uses of the Invention

Uses of the Compounds as Research Tools and Kits

Synthetic polyammonium compounds may interfere with a number of important biological processes involving protein-protein (P-P), protein-nucleic acid (P-NA) and nucleic acid-nucleic acid (NA-NA) interactions.

Synthetic polyamines of the invention could thus be of much interest as active agents or pharmacological tools in areas such as:
cell biology biophysics: effects on the cytoskeleton; cell migration, cell division;
protein-protein diseases (Alzheimer's, prions, etc.);
gene transfer, through interaction with oligo(poly)nucleotide sequences and siRNAs (small interfering RiboNucleic Acids);
developmental biology;
intercellular processes, such as: cell-to-cell communication via polyanions, such as miRNAs (microRiboNucleic Acids) or signalling agents, such as inositol phosphates;
cell-to-cell contacts, in particular, reparation of cell contacts (for instance in nerve cells), etc., and
cancer.

In a more specific aspect, the present invention relates to the use of a branched polyamine, macrocyclic polyamine or polyamine derivative of the invention as defined herein as a pharmacological or biological research tool, in particular as a cellular modulator of motility. The compound of the invention may be used as a laboratory or a research tool. The compound of the invention may also be used as a diagnosis tool. The compound of the invention may be also used in a screening or diagnosis method. Branched polyamines, macrocyclic polyamines or polyamine derivatives of the invention may allow studying the actin cytoskeleton in mobile and/or morphogenetic processes, more specifically studying cell dynamics, assembly-disassembly dynamics of actin filaments, lamellipodial growth or array in cells. The compounds of the invention may be further used for impairing, preferably for slowing down, an actin-based process in a cell. Actin-based process encompasses, without being limited to, re-organization of the Golgi apparatus, scission of tubulated membranes by WASH/Arp2/3, dendritic spine dynamics in synaptic plasticity, dynamics of immune synapse formed in T-cell activation, cell motility, cell division, intracellular transport of vesicles and cell migration. For instance, a compound of the invention may be used for promoting the growth of cytoplasmic projections, in particular lamellipodia. A compound of the invention may also be used for impairing, preferably for slowing-down, the assembly-disassembly actin dynamics in a cell.

Compounds of the invention may be also a potent pharmacological tool. They may be used to study cancers and more specifically metastasis in cancer, or to diagnose a disease, such as cancers and more specifically metastasis in cancer, or follow the impact of a therapy disease, such as cancer therapy, and more specifically therapy of metastasis in cancer. The compounds of the invention may be used as pharmacological tools in order to determine whether a disease involves a dysfunction of actin dynamics, or relies on an actin-based process such as lamellipodial growth, cell motility, or cell migration.

As used herein, an actin-based process encompasses any cellular event involving or requiring actin cytoskeleton, in particular a remodeling of the actin cytoskeleton. The remodeling of the cellular actin cytoskeleton may involve the depolymerization of preformed actin filaments, the polymerization of new actin filaments as well as the formation and/or the destruction of intracellular actin structures such as actin bundles and actin networks.

As used herein, “a compound able to impair or alter an actin-based process” refers to a compound which, when contacted with a cell, is able to modify an actin-based process in said cell as compared to that occurring in a similar cell which is not contacted with said compound. As used herein, an “impairment” or an “alteration” encompasses any type of “modifications” of the actin-based process, such that the inhibition or the blockage of the actin-based process, the slowing-down of the actin-based process, the speeding-up of the actin-based process, the increase or the decrease of the biological effects potentially triggered by the actin-cell process, as compared to those observed in a cell which has not be contacted with said compound.

In some embodiments, the compounds of the invention may impair the actin cytoskeleton per se or the remodeling of the actin cytoskeleton involved in said actin-based process.

As used herein, a compound able to impair the assembly-disassembly dynamics of actin refers to a compound able to affect the rate of assembly and/or the rate of disassembly of actin filaments, for instance by promoting or inhibiting actin nucleation, and/or by altering (e.g speeding-up or slowing down) actin polymerization on barbed or pointed end, and/or by altering (e.g. speeding-up or slowing down) depolymerization of actin filaments on barbed or pointed end. For instance, a compound of the invention may be able to slow-down the growth of actin filament on barbed end in vitro and/or may be able to decrease the destabilization of actin filament by ADF.

Preferably, the compound of the invention is able to slow-down the assembly-disassembly dynamics of actin. The ability of a compound to impair assembly-disassembly dynamics of actin may be for instance assessed as shown in the below examples, e.g. by studying the effect of the compound in acellular in vitro assay such as in N-WASP bead propulsion assay, in actin treadmilling assay or in actin polymerization assay.

The compounds of the invention are preferably used ex vivo, in particular in vitro and more preferably in cellulo. In other words, the compounds of the invention are preferably used on isolated cell, cell culture, isolated tissue or isolated organ.

However, the compounds of the invention may be also used as a pharmacological tool to study an animal model, for instance a chemical or a genetic animal model of a disease. It goes without saying that said animal is not human.

A further object of the invention is the in vitro use of the compounds of the invention for modifying a biological process selected from cell division, cell motility, and cell migration.

The invention also relates to an in vitro method for studying assembly-disassembly dynamics of actin filaments or lamellipodia, in particular lamellipodia growth, in a cell, comprising the steps of:
(a) providing a compound of the invention, in particular a compound of any one or formulae (I), (Ia), (Ib), (V) and (Va) or a derivative thereof,
(b) contacting the compound of step (a) with a cell, and
(c) observing the assembly-disassembly dynamics of actin filaments or lamellipodial growth in said cell.

Said method may further comprise comparing the cell which has been contacted with the compound of the invention in step (b) with a similar cell which has not been contacted with said compound of the invention.

In some embodiments, the compound of the invention may comprise a label moiety. Said label may be useful for performing the step (c) of observation. For instance, the compound of the invention may be selected among compounds of formula (Ic) and (Id) as defined above, such as compound C₈N₆ BPA-Cy3 .

The invention also relates to an in vitro method for altering, preferably for slowing-down an actin-based process in a cell, said method comprising contacting said cell with a compound of the invention.

In a more specific aspect, the invention also relates to an in vitro method for promoting the growth of lamellipodia and/or for altering, preferably for slowing-down, the assembly-disassembly actin dynamics in a cell, said method comprising contacting a compound of the invention, in particular a compound of any one or formulae (I), (Ia), (Ib), (V) and (Va) or a derivative thereof with said cell.

It goes without saying that the cell is contacted with said compound in conditions conducive to the growth of lamellipodia and/or to modify actin dynamics.

In another aspect, the invention also relates to an in vitro method for inhibiting motility in a cell susceptible to be motile, said method comprising contacting a compound of the invention, in particular a compound of any one or formulae (I), (Ia), (Ib), (V) and (Va) or a derivative thereof, with said cell.

A further object of the invention is an in vitro method for assessing the effect of a test compound on lamellipodia growth and/or on actin dynamics, said method comprising the steps of:
(a) contacting a cell with the test compound and optionally with a compound of the invention, and
(b) comparing lamellipodia growth and/or actin dynamics in cell of step (a) with a similar cell which has been contacted with a compound of the invention only, whereby the effect of the test compound on lamellipodia growth and/or on actin dynamics is determined.

The uses and the methods of the invention may be performed on any type of cell of interest. The cell is selected depending on the aim of the use or the method to implement. For instance, the cell may derive from fibroblast, osteosarcoma or adenocarcinoma cell lines, stem cells, epithelial cells, or endothelial cells. The methods and the uses of the invention are preferably not performed on human embryo or human embryogenic cells. However, the methods and uses of the invention may be performed on non-human embryogenic cell.

Particular compounds of interest for implementing the uses and the methods of the invention as disclosed herein are compounds of formula (I), (Ia), (Ib), (V), (Va), and (VI). Particular compounds are
N,N,N′,N′-[tetrakis(aminopropyl)trimethylenediamine (C₃N₆BPA):
N,N,N,′N′-tetrakis(aminopropyl)decamethylenediamine (C₁₀N₆ BPA):
N,N,N′,N′-[tetrakis(aminopropyl)nonamethylenediamine (C₉N₆BPA):
N,N,N′,N′-[tetrakis(aminopropyl)octamethylenediamine (C₈N₆ BPA):
N,N,N′,N′-[tetrakis(aminopropyl)heptamethylenediamine (C₇N₆ BPA):
N,N,N′,N′-[tetrakis(aminopropyl)hexamethylenediamine (C₆N₆ BPA):
C₈N₁₄BPA,

as well as salts and derivative thereof.

Other particular compounds of interest for implementing the uses and the methods of the invention as disclosed herein are compounds of formula (Ic) ad (Id).

In preferred embodiments, the methods and uses according to the invention are performed with branched polyamines of the invention, for instance branched polyamines of formula (I), (Ia), (Ib) (Ic) or (Id).

In some embodiments, the compound of the invention may be conjugated to a label enabling the detection, such as a fluorophore. In some preferred embodiments, the label is linked to an amino group of the compound. Preferably, the compound of the invention is selected among branched polyamines of formula (Ic) or (Id). An example of such fluorophore-containing branched polyamine of the invention is

Cy-3 dye labeled—N,N,N′,N′-[tetrakis(aminopropyl)octamethylenediamine (C₈N₆ BPA-Cy3).

Compounds of the invention comprising a label, in particular a fluorophore moiety, may be used as markers of actin, and more precisely as imaging tool for the visualization of actin-based structures in vitro. Said compounds may be used for coating or labelling actin and actin-based structures such as actin filaments, actin network and actin bundles. For instance, compounds of the invention comprising a label may be used in cytology for visualizing actin-based structure in cells.

Another object of the invention is a kit, preferably for implementing any one of the methods or uses as described herein, and comprising a compound of the invention as defined herein, in particular a compound of formulae (I), (Ia), (Ib), (Ic), (Id), (V), (Va) or (VI). Said kit may further comprise:
reagents such as a buffer, culture medium and the like, and/or
a cell or a cell culture and/or
device such as microplates, detection means and the like, and/or
an actin dynamics modulator or any other compound able to modulate or play a role in motility cells, in actin polymerization or in actin filament stability, preferably in an additional compartment, preferably said compoundis selected in the group consisting of latrunculin A and B, cytochalasin D, jasplakinolide, wiskotatin, CK666, SMIFH2, blebbistatin, ML-7, Y27632, ADF, Arp2/3, an actin nucleation agents such as ActA, IscA, RickA, WASp, N-WASP, pWa and SCAR-WAVE proteins, formins, spire, profilin, gelsolin, capping proteins, a cross-linking protein such as alpha-actinin, fascin, EF-1, Scruin, villin, dematin, fimbrin, spectrin, dystrophin, ABP 120, filamin, and one of their mixtures and/or
a detection compound, in particular a compound for the detection of actin structures such as a labeled phalloidin (e.g. alexa 488 phalloidin) or a labeled antibody directed against actin, and/or
written instructions, for instance, relating to the running conditions for implementing a method or an use of the invention.

Another object of the invention relates to a kit comprising at least one branched polyamine, macrocyclic polyamine or polyamine derivative as defined herein and optionally monomeric actin, and/or actin seeds and/or actin filaments, where preferably each of the compounds comprised therein is located in different compartments of the kit. The kit can further include, if need be, controls and/or instructions.

Said kit may be useful for preparing actin filaments or actin-based structures in vitro.

Said kit may further comprise at least one additional compound such as actin dynamics modulator or any other compound able to modulate or play a role in motility cells, actin polymerization and/or actin filament stabilization preferably in an additional compartment, preferably said compound, including the actin dynamics modulator, is selected in the group consisting of latrunculin A and B, cytochalasin D, jasplakinolide, wiskotatin, CK666, SMIFH2, blebbistatin, ML-7, Y27632, ADF, Arp2/3, an actin nucleation agent such as ActA, IscA, RickA, WASp, N-WASP, pWa and SCAR-WAVE proteins, formins, spire, profilin, gelsolin, capping proteins, a cross-linking protein such as alpha-actinin, fascin, EF-1, Scruin, villin, dematin, fimbrin, spectrin, dystrophin, ABP 120, filamin, and one of their mixtures. Preferably, the additional compound is selected from formin, spire, Arp2/3, an actin nucleation agent such as ActA, IscA, RickA, WASp, N-WASP, pWa and SCAR-WAVE proteins, profilin, gelsolin, a capping protein and mixture thereof. Said kit may also contain ATP and a source of divalent cation such as MgCl₂ or CaCl₂.

In preferred embodiments, the kits of the invention as defined above comprise a branched polyamines of the invention, for instance a branched polyamine of formula (I), (Ia), (Ib) (Ic) or (Id). In some embodiments, the kits of the invention do not contain a macrocyclic polyamine of the invention, in particular a compound of formula (V), (Va) or (VI).

The invention also relates to the use of a compound of the invention for promoting actin polymerization, in particular actin bundles in vitro.

Typically, the compound of the invention may be added into a solution containing actin monomers, ATP, divalent cations such as Ca2+, and optionally at least one actin filament or actin seed, whereby the formation of actin bundles in the solution is promoted.

In some embodiments, the solution may further contain at least one actin dynamic modulator as described above, preferably selected from the group consisting of formin, spire, Arp2/3, an actin nucleation agent such as ActA, IscA, RickA, WASp, N-WASP, pWa and SCAR-WAVE proteins, profiling, gelsolin, a capping protein and mixtures thereof. Preferred compounds of the invention are branched polyamines of formula (I), (Ia), (Ib), (Ic) or (Id). Said use is preferably performed in the absence of cell, namely in an acellular medium.

The invention also relates to a method for promoting actin polymerization, in particular actin bundle, said method comprising added a compound of the invention, preferably a branched polyamine of the invention into a medium comprising actin monomers, ATP, divalent cations and optionally actin filaments or actin seeds. The solution may further comprise one or several actin dynamic modulator as defined above. In preferred embodiments, the compound of the invention is a branched polyamine of formula (I), (Ia), (Ib), (Ic) or (Id).

Pharmaceutical Compositions and Therapeutic Uses of the Compounds According to the Invention

The present invention also relates to a compound of the invention, in particular a compound of anyone of formulae (I), (Ia), (Ib), (V), or (Va), or any polyamine derivative of the invention, as well as any particular compound disclosed herein, for use as a drug, in particular, for use in the treatment of a disease involving protein-protein interaction, protein-nucleic acid interaction and/or nucleic acid-nucleic acid interaction.

The present invention further relates to the use of a compound of the invention for the manufacture of a medicament, preferably for treating of a disease involving protein-protein interaction, protein-nucleic acid interaction and/or nucleic acid-nucleic acid interaction

A further object of the present invention is a method for treating a patient comprising administering an effective amount of a compound of the invention to the said patient. More precisely, the present invention relates to a method for treating a disease involving protein-protein interaction, protein-nucleic acid interaction and/or nucleic acid-nucleic acid interaction.

Said diseases may involve biological compounds chosen (i) among proteins comprising at least one aspartic acid residue, phosphate site and/or glutamic acid residue, and/or among (ii) molecules comprising at least one nucleotide and/or oligonucleotide.

Diseases of interest include, but are not limited to, diseases or disorders implying the cytoskeleton, cell migration, cell division, neurodegenerative diseases, such as Alzheimer's disease, diseases implying prions, gene transfer, through interaction with oligo(poly)nucleotide sequences, siRNAs, developmental biology, miRNAs, reparation of cell contacts and cancer, more specifically metastasis.

Neurodegenerative diseases, include without being limited to, Alzheimer's disease, tauopathies, and polyglutamine diseases such as Huntington's disease or spinocerebellar ataxia.

In some preferred embodiments, the disease or the disorder is associated with a dysfunction of cytoskeleton, in particular a dysfunction or dysregulation of actin dynamics, and/or with remodeling of actin cytoskeleton.

As used herein, a disease or a disorder is associated with, or involved, a dysfunction of cytoskeleton means that the onset, the development and/or the spread of the disease rely on a dysfunction of cytoskeleton or that the disease causes a dysfunction of the cytoskeleton. In other words, the dysfunction of the cytoskeleton may be a factor promoting the onset and/the development of said disease, or may be provoked by the development of the disease.

In some other or additional embodiments, the onset, the development and/or the spread of said disease or disorder are based on an cellular actin-based process such as cell division, cell motility, or cell migration.

In some particular embodiments, the disease to treat is selected from Alzheimer's disease, cancer, in particular metastasis.

In a preferred embodiment, the therapeutic method or the therapeutic use of the invention is for preventing or treating metastasis. For instance, the compounds of the invention may be used for decreasing, blocking, or preventing metastasis from a primary cancer. The compounds of the invention may be used for preventing cell migration or cell motility, in particular, for preventing the migration and/or the spreading of metastasis.

In a more particular aspect, the therapeutic method or use of the invention is based on a compound of formula (Ia), (IIb), (Va) or (VI).

Preferred compounds of the invention are among other:
N,N,N′,N′-[tetrakis(aminopropyl)trimethylenediamine (C₃N₆BPA):
N,N,N,′N′-tetrakis(aminopropyl)decamethylenediamine (C₁₀N₆ BPA):
N,N,N′,N′-[tetrakis(aminopropyl)nonamethylenediamine (C₉N₆BPA):
N,N,N′,N′-[tetrakis(aminopropyl)octamethylenediamine (C₈N₆ BPA):
N,N,N′,N′-[tetrakis(aminopropyl)heptamethylenediamine (C₇N₆ BPA):
N,N,N′,N′-[tetrakis(aminopropyl)hexamethylenediamine (C₆N₆ BPA):
C₈N₁₄BPA,
The compound of formula (VI)

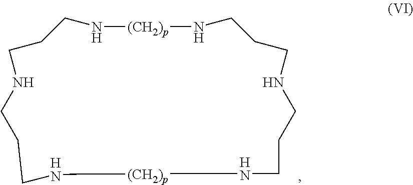

wherein p is 7; and
pharmaceutically salts thereof.

A “therapeutically effective amount” of the compound of the invention to administer to the patient refers to the amount of the compound of the invention which prevents, removes, slows down the disease or the disorder to treat. Said amount may also refer to the amount of the compound of the invention capable for reducing or delaying one or several symptoms caused by or associated with the disease or the disorder to treat in said patient. The effective amount, and more generally the dosage regimen, of the compound of the invention and pharmaceutical compositions thereof may be easily determined and adapted by the one skilled in the art. An effective dose can be determined by the use of conventional techniques and by observing results obtained under analogous circumstances. The therapeutically effective dose of the compound of the invention may vary depending on such factors as the pathological condition to be treated (including prevention), the method of administration, any co-therapy involved, the patient's age, weight, general medical condition, medical history, etc. Typically, the amount of the compound to be administrated to a patient may range from about 0.01 mg/day/kg to 50 mg/day/kg of body weight, preferably from 0.1 mg/day/kg to 25 mg/day/kg of body weight. For example, for a patient having a body weight of 60 kg, the daily dosage for the compound of the invention ranges from 0.6 mg to 3 g, preferably from 6 mg to 1.5 g.

The compounds of the invention may be administered by various routes, including, but not limited to, oral, subcutaneous, intravenous, parenteral, intranasal, intraortical, intraocular, rectal, vaginal, transdermal, topical (e.g., gels), intraperitoneal, or intramuscular route.

As explained above, the compounds of the invention may be used as therapeutic agents. Thus, another aspect of the invention is a pharmaceutical composition comprising a compound of the invention, namely a macrocylic polyamine, a branched polyamine or a polyamine derivative as defined above, and a pharmaceutically acceptable excipient.

Preferably, the compound of the invention is present in the pharmaceutical composition as the active ingredient. However, in some alternate embodiments, the compound of the invention may be present as an agent for complexing a molecule of therapeutic interest such as a nucleic acid.

In some particular embodiments, the compound of the invention present within the pharmaceutical composition is selected among any compounds of formula (I), (Ia), (Ib), (V) and (Va) as disclosed above.

In some more particular embodiments, the pharmaceutical composition comprises a compound of the invention selected from
N,N,N,N-[tetrakis(aminopropyl)trimethylenediamine (C₃N₆BPA):
N,N,N,′N′-tetrakis(aminopropyl)decamethylenediamine (C₁₀N₆ BPA):
N,N,N′,N′-[tetrakis(aminopropyl)nonamethylenediamine (C₉N₆BPA):
N,N,N′,N′-[tetrakis(aminopropyl)octamethylenediamine (C₈N₆ BPA):
N,N,N′,N′-[tetrakis(aminopropyl)heptamethylenediamine (C₇N₆ BPA):
N,N,N′,N′-[tetrakis(aminopropyl)hexamethylenediamine (C₆N₆ BPA):
C₈N₁₄BPA,
The compound of formula (VI)

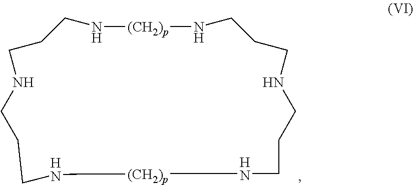

wherein p is 7; and
pharmaceutically salts thereof.

The pharmaceutical composition of the invention may be formulated according to standard methods such as those described in Remington: The Science and Practice of Pharmacy (Lippincott Williams & Wilkins; Twenty first Edition, 2005). Pharmaceutically acceptable excipients that may be used are described, for example, in the Handbook of Pharmaceuticals Excipients, American Pharmaceutical Association (Pharmaceutical Press; 6th revised edition, 2009). The pharmaceutical composition of the invention may be obtained by admixing a compound of the invention with an appropriate degree of purity with at least one pharmaceutically acceptable excipient such as (a) diluents such as for example, starch, lactose, sucrose, glucose, mannitol, calcium carbonate, sodium carbonate, calcium phosphate, sodium phosphate and microcrystalline cellulose; (b) binders such as, carboxymethylcellulose, gelatin, polyvinylpyrrolidone, sucrose; (c) humectants such as glycerol; (d) disintegrating agents such as maize starch and sodium croscarmellose; (e) solution retarders, (f) wetting agents, such as glycerol monostearate; (h) adsorbents such as kaolin and bentonite; (g) lubricants such as talc, calcium stearate, magnesium stearate, solid polyethylene glycols, sodium lauryl sulfate, (i) antioxidant agents, (j) buffering agents such as sodium citrate or sodium phosphate, (k) preservatives, (l) flavours and perfumes, etc.

It goes without saying that (i) the excipient(s) to be combined with (ii) the active ingredient may vary upon (i) the physico-chemical properties including the stability of the said active ingredient, (ii) the pharmacokinetic profile desired for said active ingredient, (iii) the galenic form and (iv) the route of administration.

The pharmaceutical composition may be in the form of an uncoated or coated tablet, a capsule, a pill, granules, a powder, an emulsion, a suspension, a solution, a syrup, a cream, a gel, an ointment, a suppository, and the like.

The pharmaceutical composition may comprise:
from 0,01% to 50% by weight of a compound of the invention, and
from 50% to 99.99% by weight of excipients,

the percentage being expressed as compared to the total weight of the composition.

Preferably, the pharmaceutical composition may comprise:
from 0,1% to 25% by weight of a compound of the invention, and
from 75% to 99,9% by weight of excipients.

The amount of the compound of the invention in the pharmaceutical composition may depend on the form of the said composition.

The compounds and the pharmaceutical compositions of the invention may be administered by any conventional route, including by oral route by parenteral route and by topical route.

Further aspects and advantages of the present invention will be disclosed in the following experimental section, which should be regarded as illustrative and not limiting the scope of the present application.

### EXAMPLES

### Example 1

### Preparation of the Compounds of the Invention

A. Chemical Synthesis of the Branched Polyamines

The branched polyamines (BPAs) were synthesized by a sequence involving: 1) treatment of terminal linear aliphatic diamines with acrylonitrile to give branched polynitriles, followed by 2) reduction of the nitrile groups to give the corresponding polyamines.

General Procedure for the Preparation of Branched Polynitriles

The general procedure for the preparation of branched polynitriles, the building blocks for preparing the branched polyamines of the invention, was as follows:

Acrylonitrile (5.0 equiv.) was added dropwise to the corresponding diamine (1.0 eq) dissolved in H₂O at room temperature. The resulted reaction mixture was warmed up to 50° C. and stirred for 3 h. The temperature was then increased to 80° C. and additional acrylonitrile (5.0 equiv.) was added dropwise. After 12 h, the reaction mixture was cooled to room temperature, the layers were separated and the aqueous layer was extracted with CH₂Cl₂ (3×100 mL). The combined organic layers were dried over Na₂SO₄, filtered and concentrated in vacuo. The crude product was passed through a pad of silica (eluant: CH₂Cl₂/EtOAc) to afford the corresponding nitriles as colorless oils.

### Synthesis N,N,N′,N′-[tetrakis(cyanoethyl)trimethylenediamine

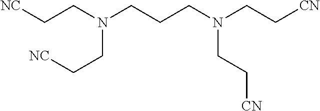 [SMILES: N#CCCN(CCC#N)CCCN(CCC#N)CCC#N]

1,3-Propane diamine (2.0 g, 27.0 mmol, 1 equiv.), H₂O (80 ml), acrylonitrile (8.8 ml×2=17.6 ml, 270 mmol, 5×2 equiv.); extraction silica (eluant: CH₂Cl₂/EtOAc , 7/3; R_{f}=0.3) Yield: 7.1 g (92%);

¹H-NMR (400 MHz, CDCl₃): δ=2.83 (t, J=6.6 Hz, 8H), 2.63 (t, J=6.7 Hz, 4H), 2.49 (t, J=6.5 Hz, 8H), 1.63 (qn, J=6.7 Hz, 2H); ¹³C-NMR (100 MHz, CDCl₃): δ=118.8, 50.5, 49.2, 25.3, 16.7; HRMS (ESI): m/z: calcd for C₁₅H₂₂N₆: 287.191 [M+1]⁺; found: 287.190.

### Synthesis N,N,N′,N′-[tetrakis(cyanoethyl)hexamethylenediamine

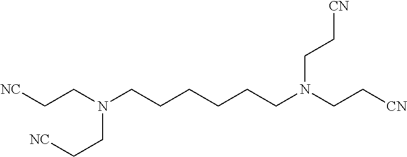 [SMILES: N#CCCN(CCC#N)CCCCCCN(CCC#N)CCC#N]

1,6-hexanediamine (4.0 g, 34.4 mmol, 1 equiv.), H₂O (100 ml), acrylonitrile (11.2 ml×2=22.4 ml, 344 mmol, 5×2 equiv.); silica (Eluant: CH₂Cl₂/EtOAc, 8/2; R_{f}=0.25) Yield: 10.2 g (94%);

¹H-NMR (400 MHz, CDCl₃): δ=2.81 (t, J=6.7 Hz, 8H), 2.50 (t, J=7.0 Hz, 4H), 2.44 (t, J=6.7 Hz, 8H), 1.48-1.40 (m, 4H), 1.34-1.30 (m, 4H); ¹³C-NMR (100 MHz, CDCl₃): δ=118.6, 53.1, 49.4, 27.1, 26.6, 16.8; HRMS (ESI): m/z: calcd for C₁₈H₂₈N₆: 329.238 [M+1]⁺; found: 329.238.

### Synthesis of N,N,N′,N′-[tetrakis(cyanoethyl)heptamethylenediamine

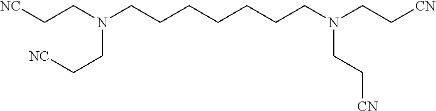 [SMILES: N#CCCN(CCC#N)CCCCCCCN(CCC#N)CCC#N]

1,7-heptanediamine (5.0 g, 38.4 mmol, 1 equiv.), H₂O (110 ml), acrylonitrile (12.5 ml×2=25.1 ml, 384 mmol, 10 equiv); silica (Eluant: CH₂Cl₂/EtOAc, 8/2; R_{f}=0.3) Yield: 12.6 g (96%);

¹H-NMR (400 MHz, CDCl₃): δ=2.83 (t, J=6.8 Hz, 8H), 2.50 (t, J=7.1 Hz, 4H), 2.45 (t, J=6.8 Hz, 8H), 1.50-1.37 (m, 4H), 1.30 (br. s, 6H); ¹³C-NMR (100 MHz, CDCl₃): δ=118.6, 53.3, 49.5, 27.1, 26.8, 16.8; HRMS (ESI): m/z: calcd for C₁₉H₃₀N₆: 343.253 [M+1]⁺; found: 343.253.

### Synthesis of N,N,N′,N′-[tetrakis(cyanoethyl)octamethylenediamine

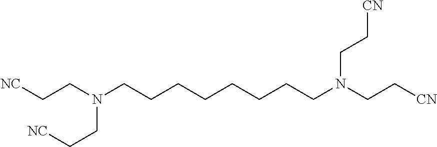 [SMILES: N#CCCN(CCC#N)CCCCCCCCN(CCC#N)CCC#N]

1,8-octane diamine (5.0 g, 34.6 mmol, 1 equiv.), H₂O (100 ml), acrylonitrile (11.3 ml×2=22.6 ml, 346 mmol, 10 equiv); silica (Eluant: CH₂Cl₂/EtOAc, 9/1; R_{f}=0.32) Yield: 11.1 g (90%);

¹H-NMR (400 MHz, CDCl₃): δ=2.83 (t, J=6.7 Hz, 8H), 2.50 (t, J=7.3 Hz, 4H), 2.45 (t, J=6.7 Hz, 8H), 1.46-1.39 (m, 4H), 1.29 (br. s, 8H); ¹³C-NMR (100 MHz, CDCl₃): δ=118.6, 53.3, 49.5, 29.2, 27.2, 26.8, 16.9; HRMS (ESI): m/z: calcd for C₂₀H₃₂N₆: 357.269 [M+1]⁺; found: 357.268.

### Synthesis of N,N,N′,N′-[tetrakis(cyanoethyl)nonamethylenediamine

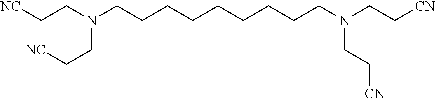 [SMILES: N#CCCN(CCC#N)CCCCCCCCCN(CCC#N)CCC#N]

1,9-nonanediamine (5.0 g, 31.6 mmol, 1 equiv.), H₂O (90 ml), acrylonitrile (10.3 ml×2=20.6 ml, 316 mmol, 10 equiv); silica (Eluant: CH₂Cl₂/EtOAc, 9/1; R_{f}=0.36) Yield: 10.8 g (92%);
¹H-NMR (400 MHz, CDCl₃): δ=2.82 (t, J=6.8 Hz, 8H), 2.49 (t, J=7.3 Hz, 4H), 2.44 (t, J=6.8 Hz, 8H), 1.46-1.37 (m, 4H), 1.26 (br. s, 10H); ¹³C-NMR (100 MHz, CDCl₃): δ=118.6, 53.3, 49.4, 29.3, 29.1, 27.1, 26.8, 16.8; HRMS (ESI): m/z: calcd for C₂₁H₃₄N₆: 371.284 [M+1]⁻; found: 371.284.

### Synthesis of N,N,N′,N′-[tetrakis(cyanoethyl)decamethylenediamine

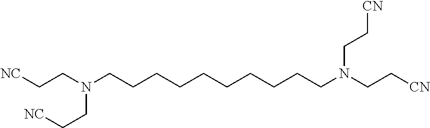 [SMILES: N#CCCN(CCC#N)CCCCCCCCCCN(CCC#N)CCC#N]

1,10-decane diamine (5.0 g, 29.0 mmol, 1 equiv.), H₂O (85 ml), acrylonitrile (9.5 ml×2=19.0 ml, 290 mmol, 10 equiv); silica (Eluant: CH₂Cl₂/EtOAc, 9/1; R_{f}=0.3) Yield: 10.6 g (95%);

¹H-NMR (400 MHz, CDCl₃): δ=2.84 (t, J=6.8 Hz, 8H), 2.51 (t, J=7.3 Hz, 4H), 2.45 (t, J=6.8 Hz, 8H), 1.48-1.38 (m, 4H), 1.27 (br. s, 12H); ¹³C-NMR (100 MHz, CDCl₃): δ=118.5, 53.4, 49.6, 29.4, 29.3, 27.2, 26.9, 16.9; HRMS (ESI): m/z: calcd for C₂₂H₃₆N₆: 385.300 [M+1]⁻; found: 385.300.

2^{nd} Generation Cyanation of C-8 Branched Polyamine:

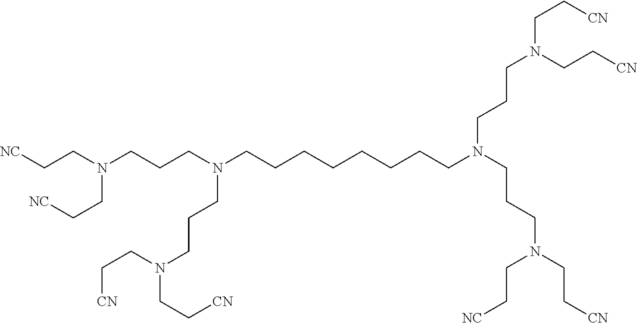 [SMILES: N#CCCN(CCC#N)CCCN(CCCCCCCCN(CCCN(CCC#N)CCC#N)CCCN(CCC#N)CCC#N)CCCN(CCC#N)CCC#N]

N,N,N′,N′-[tetrakis(aminopropyl)octamethylenediamine (500 mg, 1.40 mmol, 1 equiv.), H₂O (10 ml), acrylonitrile (0.92 ml×2 =1.83 ml, 28.0 mmol, 20 equiv); silica (Eluant: CH₂Cl₂/EtOAc, 9/1; R_{f}=0.15) Yield: 1.1 g (96%);

¹H-NMR (400 MHz, CDCl₃): δ=2.84 (t, J=6.7 Hz, 16H), 2.56 (t, J=7.0 Hz, 8H), 2.48 (t, J=6.7 Hz, 16H), 2.45 (t, J=7.5 Hz, 8H), 2.37 (t, J=7.4 Hz, 4H), 1.62-1.55 (m, 8H), 1.44-1.36 (m, 4H), 1.32-1.22 (br. m, 8H); ¹³C-NMR (100 MHz, CDCl₃): δ=118.7, 54.0, 51.6, 51.4, 49.6, 29.7, 27.6, 27.0, 25.1, 16.9; HRMS (ESI): m/z: calcd for C₄₄H₇₂N₁₄: 797.606 [M+1]⁻; found: 797.605.

General Procedure for the Preparation of Amines

The general procedure for the preparation of branched polyamines was as follows: Nitrile compound (1.0 equiv) was dissolved in a mixture of abs. EtOH and THF. Raney-nickel (8.2 equiv.) as a 50% suspension in water was added together with 2 N aq. NaOH solution and the reaction mixture was degassed and stirred under 1 atm of H₂ at room temperature for 24 h. The mixture was filtered off through a pad of celite, washed with EtOH (50 ml) and the solvents were removed in vacuo. The residue was dissolved in a mixture of water (50 mL) and CHCl₃ (100 mL), the layers were separated and the aqueous layer was extracted with CHCl₃ (4×100 mL). The combined organic layers were dried over Na₂SO₄, filtered and the solvent evaporated in vacuo. Amines were obtained as light yellow oils.

The hydrochloride salts of corresponding amines were prepared as follows: to the amine (1 mmol) in hot methanol (10 mL), was added 6M HCl (3 mL) and hot EtOH (10 mL) and the mixture was left at room temperature till the precipitate was formed. The precipitate was filtered off, washed with cold ethanol and dried in vacuo.

### Preparation of N,N,N′,N′-[tetrakis(aminopropyl)trimethylenediamine (C₃N₆BPA)

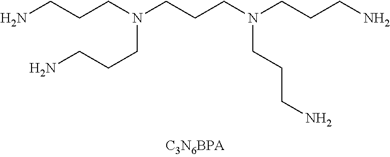 [SMILES: NCCCN(CCCN)CCCN(CCCN)CCCN]

N,N,N′,N′-[tetrakis(cyanoethyl)trimethylenediamine (2.0 g, 7.00 mmol, 1 equiv.), EtOH (160 ml), THF (40 ml) Raney-Nickel (6.7 g, 57.2 mmol, 8.2 equiv.) as a 50% suspension in water, 2 N NaOH soln. (40 ml) Yield: 2.0 g (95%);

¹H-NMR (400 MHz, CDCl₃): δ=2.68 (t, J=6.9 Hz, 8H), 2.41 (t, J=7.1 Hz, 8H), 2.36 (t, J=7.4 Hz, 4H), 1.54 (qn, J=6.9 Hz, 10H), 1.47 (br.s, 8H); ¹³C-NMR (100 MHz, CDCl₃): δ=52.2, 51.8, 40.6, 30.8, 24.5; HRMS (ESI): m/z: calcd for C₁₅H₃₈N₆: 303.316 [M+1]⁺; found: 303.316.

### Preparation of N,N,N′,N′-[tetrakis(aminopropyl)hexamethylenediamine C₆N₆BPA

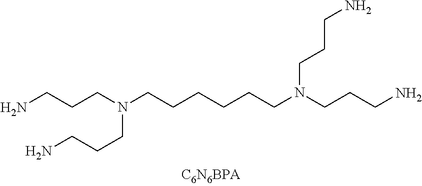 [SMILES: NCCCN(CCCN)CCCCCCN(CCCN)CCCN]

N,N,N′,N′-[tetrakis(cyanoethyl)hexamethylenediamine (2.0 g, 6.08 mmol, 1 equiv.), EtOH (140 ml), THF (35 ml) Raney-Nickel (5.83 g, 49.8 mmol, 8.2 equiv.) as a 50% suspension in water, 2 N NaOH soln. (35 ml) Yield: 1.9 g (91%);

¹H-NMR (400 MHz, D₂O): δ=3.45-3.33 (br m, 8H), 3.36-3.29 (m, 4H), 3.24-3.16 (m, 8H), 2.24 (br m, 8H), 1.85 (br m, 4H), 1.53 (br m, 4H), NH₂ signal not visible; ¹³C-NMR (100 MHz, D₂O): δ=53.2, 50.2, 36.6, 25.4, 23.1, 21.7; HRMS (ESI): m/z: calcd for C₁₈H₄₄N₆: 345.363 [M+1]⁻; found: 345.363.

### Preparation of N,N,N′,N′-[tetrakis(aminopropyl)heptamethylenediamine (C₇N₆BPA)

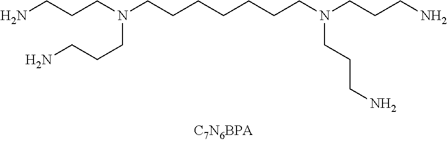 [SMILES: NCCCN(CCCN)CCCCCCCN(CCCN)CCCN]

N,N,N′,N′-[tetrakis(cyanoethyl)heptamethylenediamine (3.0 g, 8.76 mmol, 1 equiv.), EtOH (200 ml), THF (50 ml) Raney-Nickel (8.4 g, 71.8 mmol, 8.2 equiv.) as a 50% suspension in water, 2 N NaOH soln. (50 ml) Yield: 3.0 g (95%);

¹H-NMR (400 MHz, CDCl₃): δ=2.66 (t, J=6.8 Hz, 8H), 2.39 (t, J=7.1 Hz, 8H), 2.31 (t, J=7.4 Hz, 4H), 1.58-1.50 (m, 16H), 1.39-1.31 (m, 4H), 1.27-1.17 (br m, 6H); ¹³C-NMR (100 MHz, CDCl₃): δ=54.0, 51.8, 40.6, 30.8, 29.5, 27.5, 26.9; HRMS (ESI): m/z: calcd for C₁₉H₄₆N₆: 359.378 [M+1]⁺; found: 359.378.

### Preparation of N,N,N′,N′-[tetrakis(aminopropyl)octamethylenediamine (C₈N₆ BPA)

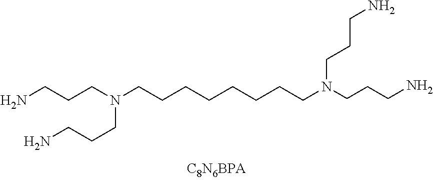 [SMILES: NCCCN(CCCN)CCCCCCCCN(CCCN)CCCN]

N,N,N′,N′-[tetrakis(cyanoethyl)octamethylenediamine (3.0 g, 8.41 mmol, 1 equiv.), EtOH (190 ml), THF (50 ml) Raney-Nickel (8.06 g, 68.9 mmol, 8.2 equiv.) as a 50% suspension in water, 2 N NaOH soln. (50 ml) Yield: 3.06 g (98%);

¹H-NMR (400 MHz, D₂O): δ=3.35-3.31 (m, 8H), 3.27-3.21 (m, 4H), 3.12 (t, J=7.8 Hz, 8H), 2.21-2.12 (m, 8H), 1.79-1.68 (m, 4H), 1.39 (br. s, 8H); ¹³C-NMR (100 MHz, D₂O): δ =53.3, 49.8, 36.5, 28.1, 25.6, 23.1, 21.6; HRMS (ESI): m/z: calcd for C₂₀H₄₈N₆: 373.394 [M+1]⁺; found: 373.394.

### Preparation of N,N,N,′N′-[tetrakis(aminopropyl)nonamethylenediamine (C₉N₆BPA)

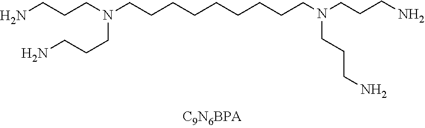 [SMILES: NCCCN(CCCN)CCCCCCCCCN(CCCN)CCCN]

N,N,N′,N′-[tetrakis(cyanoethyl)nonamethylenediamine (3.0 g, 8.1 mmol, 1 equiv.), EtOH (185 ml), THF (45 ml) Raney-Nickel (7.7 g, 66.4 mmol, 8.2 equiv.) as a 50% suspension in water, 2 N NaOH soln. (45 ml) Yield: 2.9 g (93%);

¹H-NMR (400 MHz, CDCl₃): δ=2.67 (t, J=6.8 Hz, 8H), 2.40 (t, J=7.1 Hz, 8H), 2.33 (t, J=7.4 Hz, 4H), 1.57-1.52 (m, 8H), 1.48 (br. s, 10H), 1.41-1.32 (m, 4H), 1.22 (br. s, 10H);

¹³C-NMR (100 MHz, CDCl₃): δ=54.1, 51.8, 40.7, 30.8, 29.5, 29.4, 27.5, 26.9; HRMS (ESI): m/z: calcd for C₂₁H₅₀N₆: 387.410 [M+1]⁺; found: 387.410.

### Preparation of N,N,N,′N′-tetrakis(aminopropyl)decamethylenediamine (C₁₀N₆BPA)

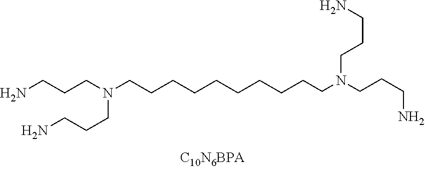 [SMILES: NCCCN(CCCN)CCCCCCCCCCN(CCCN)CCCN]

N,N,N′,N′-[tetrakis(cyanoethyl)decamethylenediamine (3.0 g, 7.80 mmol, 1 equiv.), EtOH (180 ml), THF (45 ml) Raney-Nickel (7.50 g, 63.9 mmol, 8.2 equiv.) as a 50% suspension in water, 2 N NaOH soln. (45 ml) Yield: 2.8 g (94%);

¹H-NMR (400 MHz, CDCl₃): δ=2.68 (t, J=6.8 Hz, 8H), 2.41 (t, J=7.1 Hz, 4H), 2.34 (t, J=7.5 Hz, 8H), 1.58-1.51 (m, 16H), 1.42-1.34 (m, 4H), 1.23 (br. s, 12H); ¹³C-NMR (100 MHz, CDCl₃): δ=54.1, 51.8, 40.7, 30.8, 29.5, 27.5, 26.9; HRMS (ESI): m/z: calcd for C₂₂H₅₂N₆: 401.425 [M+1]⁺; found: 401.425.

2^{nd} Generation of C-8 Amine (C₈N₁₄BPA):

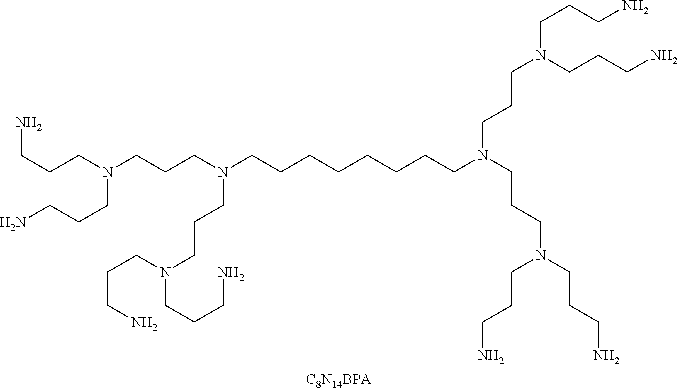 [SMILES: "C8N14BPA" ; NCCCN(CCCN)CCCN(CCCCCCCCN(CCCN(CCCN)CCCN)CCCN(CCCN)CCCN)CCCN(CCCN)CCCN]

2^{nd} generation C-8 nitrile (500 mg, 0.62 mmol, 1 equiv.), EtOH (15 ml), THF (3.5 ml) Raney-Nickel (595 m g 5.1 mmol, 8.2 equiv.) as a 50% suspension in water, 2 N NaOH soln. (3.5 ml) Yield: 460 mg (91%);

¹H-NMR (400 MHz, D₂O): δ=3.78-2.97 (m, 52H), 2.47-2.06 (m, 24H), 1.86-1.70 (M, 4H), 1.42 (br. s, 8H), 16NH₂ signals not visible; ¹³C-NMR (100 MHz, D₂O): δ=53.6, 50.1, 50.0, 49.6, 36.4, 28.3, 25.7, 23.3, 21.6, 18.9; LCMS: m/z: calcd for C₄₄H₁₀₄N₁₄: 829.857 [M+1]⁺; found: 829.857.

B. Preparation of a Cy-3 dye Labeled Branched Polyamine

Preparation of Cy3 Acid Dye

### Synthesis of N-(3-Bromopropyl)triethylammonium bromide

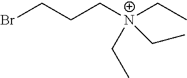

A solution of 1,3-dibromopropane (40.0 g, 198 mmol, 2 equiv.) and triethylamine (10.0 g, 99 mmol, 1 equiv.) in toluene (100 mL) were heated at 100° C. for 4 h (during this time a thick white solid gets pricipitated). The mixture was then cooled and the solid was filtered, washed with toluene and ether and dried under vacuum at 50° C. to obtain the bromopropyltriethylammonium salt (39 g, 65%) as a colorless solid;

¹H NMR (400 MHz, DMSO-d6): δ=3.62 (t, J=6.3 Hz, 2H), 3.29-3.22 (m, 8H), 2.21-2.13 (m, 2H), 1.18 (t, J=7.1 Hz, 9H); ¹³C NMR (100 MHz, DMSO-d6): δ=54.6, 52.2, 50.0, 30.7, 24.4, 7.1; HRMS (ESI): m/z: calcd for C₉H₂₁BrN⁻: 222.085 [M⁺]; found: 222.084.

### Synthesis of 1-((3-triethylammonium)propyl)-2,3,3-trimethylindolium diibromidetriethylammonium bromide

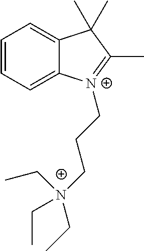 [SMILES: CC[N+](CC)(CC)CCC[N+]1=C(C)C(C)(C)c2ccccc21]

A mixture of 2,3,3-trimethylindolenine (5.0 g, 31.4 mmol, 1 equiv.) and N-(3-bromopropyl)triethylammonium bromide (9.5 g, 31.4 mmol, 1 equiv.) was heated at 140° C. for 1.5 h and cooled to room temperature (the deep red viscous melt got solidified). The solid was ground to a powder and washed with diethyl ether. The residue was purified by flash chromatography (alumina; CH₂Cl₂/MeOH, 9/1; R_{f}=0.22) to obtain the trimethylindolium dibromide salt (9.2 g, 63%) as a pale pink powder;

¹H-NMR (400 MHz, DMSO-d6): δ=8.14 (d, J=7.8 Hz, 1H), 7.87 (d, J=6.7 Hz, 1H), 7.69-7.61 (m, 2H), 4.59 (t, J=8.2 Hz, 2H), 3.28 (q, J=7.3 Hz, 6H), 2.93 (S, 3H), 2.28-2.17 (m, 2H), 1.55 (s, 6H), 1.23 (t, J=7.3 Hz, 9H); ¹³C-NMR (100 MHz, DMSO-d6): δ=197.6, 141.7, 140.9, 129.4, 128.9, 123.5, 115.4, 54.2, 52.7, 52.5, 44.3, 21.9, 20.1, 14.6, 7.3; HRMS (ESI): m/z: calcd for C₂₀H₃₄N₂^{|}:302.272 [M]; found: 302.272.

### Synthesis of 1-(5-Carboxypentyl)-2,3,3-trimethylindolium bromide

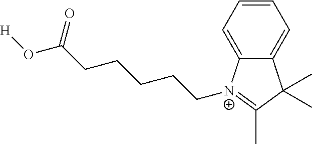 [SMILES: CC1=[N+](CCCCCC(=O)O)c2ccccc2C1(C)C]

A mixture of 2,3,3-trimethylindolenine (3.0 g, 18.8 mmol, 1 equiv.) and 6-bromohexanoic acid (5.16 g, 26.3 mmol, 1.4 equiv.) in 1,2-dichlorobenzene (50 mL) was heated at 110° C. for 12 h. The solution was cooled to room temperature and the solvent was under reduced pressure. The viscous oil obtained was diluted with diethylether/CH₂Cl₂ (1/1) and the resulted precipitate was filtered and washed with ether to obtain carboxypentyl trimenthylindolium bromide (4.5 g, 68%) as beige color solid;

¹H-NMR (400 MHz, DMSO-d6): δ=8.01-7.95 (m, 1H), 7.87-7.83 (m, 1H), 7.64-7.60 (m, 2H), 4.46 (t, J=7.8 Hz, 2H), 2.85 (s, 3H), 2.22 (t, J=7.1 Hz, 2H), 1.88-1.80 (m, 2H), 1.59-1.52 (m, 2H), 1.54 (s, 6H), 1.46-1.39 (m, 2H); ¹³C-NMR (100 MHz, DMSO-d6): δ=196.4, 174.2, 141.8, 141.0, 129.3, 128.9, 123.4, 115.4, 54.1, 47.4, 33.3, 26.9, 25.4, 23.9, 21.9, 14.0; HRMS (ESI): m/z: calcd for C₁₇H₂₄NO₂⁺: 274.180 [M]⁺; found: 274.180.

### Synthesis of 1-(5-Carboxypentyl)-2-(N-phenyl-2-aminovinyl)-3,3-dimethylindolium bromide

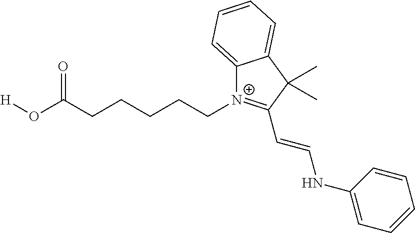 [SMILES: CC1(C)C(/C=C/Nc2ccccc2)=[N+](CCCCCC(=O)O)c2ccccc21]

A mixture of 1-(5-Carboxypentyl)-2,3,3-trimethylindolium bromide (2.0 g, 5.6 mmol, 1 equiv.) and N,N′-diphenylformamidine (2.2 g, 11.2 mmol, 2 equiv.) in acetic acid (20 mL) was heated at relux for 2.5 h. The resulted orange-red solution was cooled to room temperature and the solvent was evaporated in vacuo. The residue obtained was purified by flash chromatography (silica, CH₂Cl₂/MeOH; 9/1) to give the title compound (1.6 g, 62%) as a pink yellow orange foamy solid;

¹H-NMR (400 MHz, DMSO-d6): δ=8.67 (d, J=12.4 Hz, 1H), 7.67 (d, J=7.3 Hz, 1H), 7.54-7.43 (m, 6H), 7.34-7.27 (m, 2H), 6.28 (d, J=12.4 Hz, 1H), 4.09 (t, J=7.5 Hz, 2H), 2.22 (t, J=7.3 Hz, 2H), 1.80-1.73 (m, 2H), 1.69 (s, 6H), 1.61-1.55 (m, 2H), 1.46-1.38 (m, 2H) NH and CO₂H signals not visible; ¹³C-NMR (100 MHz, DMSO-d6): δ=177.4, 174.2, 151.8, 141.5, 140.9, 138.5, 129.7, 128.5, 126.0, 125.5, 122.5, 118.2, 91.1, 49.4, 43.9, 33.4, 27.7, 26.3, 25.7, 24.1; HRMS (ESI): m/z: calcd for C₂₄H₂₉N₂O₂^{|}: 377.222 [M]^{|}; found: 377.222.

### Synthesis of 1-(Carboxypentyl)-1′-((triethylammonium)propyl)-indocarbocyanine dibromide (Cy-3 acid dye)

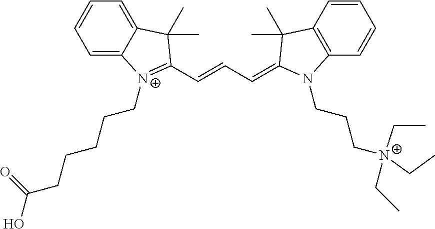 [SMILES: CC[N+](CC)(CC)CCCN1/C(=C/C=C/C2=[N+](CCCCCC(=O)O)c3ccccc3C2(C)C)C(C)(C)c2ccccc21]

A solution of 1-(5-Carboxypentyl)-2-(N-phenyl-2-aminovinyl)-3,3-dimethylindolium bromide (120 mg, 0.26 mmol, 1 equiv.) in pyridine (2.5 mL) was added acetic anhydride (0.3 mL) and the mixture was stirred for 5 mins at room temperature. 1-((3-triethylammonium)propyl)-2,3,3-trimethylindolium diibromidetriethylammonium bromide (121.2 mg, 0.26 mmol, 1 equiv.) was then added and the resulted mixture was allowed to stir for 2 h at the same temperature. The solvent was then removed under reduced pressure and the residue was dried under high vacuum. The crude product was purified by flash chromatography (neutral alumina, MeOH/CHCl₃; gradient system CHCl3 to 20% MeOH/CHCl₃) to obtain the title compound (Cy-3 acid dye) (105 mg, 54%) as a dark pink foamy powder;

¹H-NMR (400 MHz, DMSO-d6): δ=8.58 (t, J=13.5 Hz, 1H), 7.59-7.27 (m, 8H), 6.79-6.62 (m, 2H), 4.33-4.16 (m, 4H), 3.61-3.36 (m, 8H), 2.30-2.12 (m, 4H), 1.94-1.85 (m, 2H), 1.80 (s, 6H), 1.78 (s, 6H), 1.77-1.66 (m, 2H), 1.59-151 (m, 2H), 1.33 (t, J=7.3 Hz, 9H), CO₂H signals not visible; ¹³C-NMR (100 MHz, DMSO-d6): δ=176.9, 175.3, 152.4, 143.1, 143.0, 142.4, 141.9, 130.2, 130.1, 127.3, 126.6, 123.7, 123.5, 112.9, 111.7, 104.8, 103.4, 55.0, 54.3, 51.0, 50.4, 45.3, 41.6, 38.4, 28.5, 28.1, 27.7, 27.3, 26.4, 21.1, 7.7; HRMS (ESI): m/z: calcd for C₃₈H₅₅N₃O₂⁺: 585.428 [M]⁺; found: 585.428.

### Preparation of Cy-3 Dye Labeled—N,N,N′,N′-[tetrakis(aminopropyl)octamethylenediamine (C₈N₆ BPA-Cy3)

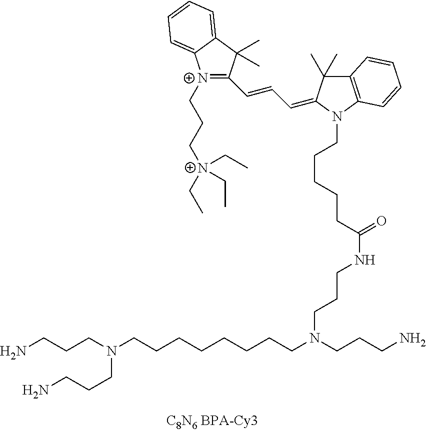 [SMILES: CC[N+](CC)(CC)CCC[N+]1=C(/C=C/C=C2/N(CCCCCC(=O)NCCCN(CCCN)CCCCCCCCN(CCCN)CCCN)c3ccccc3C2(C)C)C(C)(C)c2ccccc21]

To a solution of Cy-3 acid (30 mg, 0.05 mmol, 1 equiv.) and TSTU (O—(N-Succinimidyl)-1,1,3,3-tetramethyluronium tetrafluoroborate) (24 mg, 008 mmol, 1.6 equiv.) in DMF (1 mL) was added triethylamine (7.0 μL, 0.05 mmol, 1 equiv.). The mixture was stirred at room temperature for 1 h. The above mixture was added to a stirred solution of N,N,N,N′-[tetrakis(aminopropyl)octamethylenediamine salt (59 mg, 0.1 mmol, 2 equiv.) and Na₂CO₃ (106 mg, 1.0 mmol, 10 equiv.) in H₂O (2 ml) and the reaction mixture was stirred for 15 h. The solvent was evaporated and the residue was purified by HPLC to obtain Cy-3 dye labeled 1^{st} generation C-8 BPA;

¹H-NMR (400 MHz, D₂O): δ=8.57 (t, J=13.4 Hz, 1H), 7.59 (t, J=6.9 Hz, 2H), 7.51-7.46 (m, 2H), 7.40-7.28 (m, 4H), 6.41 (d, J=13.7 Hz, 1H), 6.29 (d, J=13.1 Hz, 1H), 4.16 (t, J=7.0 Hz, 2H), 4.10 (t, J=7.0 Hz, 2H), 3.31-3.06 (m, 27H), 2.31-2.20 (m, 4H), 2.16-2.05 (m, 6H), 1.93-1.83 (M, 5H), 1.77 (s, 6H), 1.75 (s, 6H), 1.68-1.60 (m, 6H), 1.42-1.28 (m, 10H), 1.21 (t, J=7.0 Hz, 9H); ¹³C-NMR (100 MHz, D₂O): δ=176.9, 176.3, 174.0, 151.2, 141.9, 141.8, 141.4, 140.6, 128.8, 1288.6, 125.9, 125.2, 122.6, 122.4, 117.8, 114.9, 111.8, 110.4, 103.4, 101.2, 53.4, 53.2, 53.0, 52.9, 50.1, 50.4, 49.8, 49.7, 49.0, 44.1, 40.0, 36.4, 36.0, 35.3, 28.3, 27.5, 27.0, 26.6, 25.7, 25.5, 25.0, 23.3, 23.2, 21.6, 19.6, 6.6; LCMS: m/z: calcd for C₅₈H₁₀₁N₉O₂⁺: 939.812 [M]⁺; found: 939.812.

C. Preparation of Macrocyclic Polyamine

The macrocyclic polyamine C₇N₆ MPA was obtained following the synthetic methodology described in Hosseini et Lehn³⁷.

### Example 2

Summary

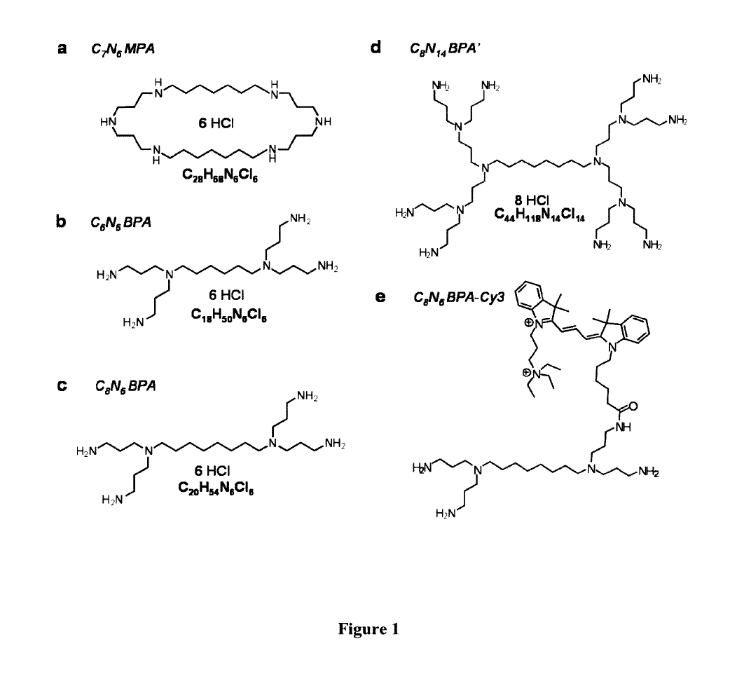

Cellular protrusions involved in motile processes are driven by site-directed assembly of actin filaments in response to Rho-GTPase signaling. So far only chemical compounds depolymerizing actin or stabilizing filaments, inhibiting N-WASP or Arp2/3 or formins have been used to eliminate the formation of protrusions, while Rho-GTPase dominant positive strategies have been designed to stimulate protrusions. The polyamines (macrocyclic and branched acyclic) as depicted in FIG. 1 were shown to enter the cell and to induce specific growth of actin-enriched lamellipodia within minutes. The largest increase in cell area was obtained with micromolar amounts of a branched polyamine harboring an 8 carbon chain. These polyamines specifically target actin both in vitro and in vivo. Analysis of their effects on filament assembly dynamics and its regulation indicates that the polyamines act by slowing down filament dynamics and by enhancing actin nucleation. These compounds provide new opportunities to study the actin cytoskeleton in motile and morphogenetic processes.

Introduction

Actin is involved in a variety of motile processes including cell migration, cell division, wound healing, synaptic plasticity, immune response and host response to pathogens. The set of chemical agents available to characterize the role of actin dynamics in such processes is limited; latrunculin A and B are known to promote filament depolymerization by sequestering G-actin; cytochalasin D inhibits actin polymerization; jasplakinolide stabilizes filaments and blocks assembly dynamics¹. More recent drug screening experiments have identified chemicals specifically inhibiting the protein machineries that control initiation of filaments, such as wiskostatin, an inhibitor of N-WASP², CK666, an inhibitor of Arp2/3³ and SMIFH2, an inhibitor of formins⁴. Alternatively, to stimulate actin assembly directly, thus avoiding pleiotropic effects of G-protein signaling, the Inventors focused on polyamines, as macrocyclic polyamines (MPAs) have been shown to induce massive assembly of G-actin in large structures at low ionic strength, thus being coined “superpolyamines”⁵.

To extend these observations further, taking into account the structural characteristics of these MPAs, the Inventors designed acyclic branched polyamines (BPAs)⁶ intended to mimic the features of MPAs. Said acyclic branched polyamines present a related pattern of two triamino-subunits separated by a single polymethylene chain and are of much greater synthetic accessibility, being thus more suitable for the exploration of structural diversity.

Results

Polyamine Compounds

Four compounds with different structural features were designed and synthesized (see Example 1 and FIG. 1): a macrocyclic polyamine, presenting two dipropylene triamine subunits linked by two aliphatic 7 carbon chains (C₇N₆ MPA) (FIG. 1a); two branched polyamines containing two dipropylene triamine groups connected through the central nitrogen by a hexamethylene chain (C₆N₆ BPA) (FIG. 1b) or a longer octamethylene chain (C₈N₆ BPA) (FIG. 1c), and another branched polyamine in which a chain of 8 methylene groups connects two terminal subunits bearing additional branches (C₈N₁₄ BPA′) (FIG. 1d) to increase the density of amine groups. Additionally, a fluorescent derivative of C₈N₆ BPA incorporating a Cy3 dye group C₈N₆ BPA-Cy3 was synthesized to allow for optical detection (FIG. 1e). The synthesis of these compounds is described in Example 1.

Universal Promotion of Lamellipodial Protrusions

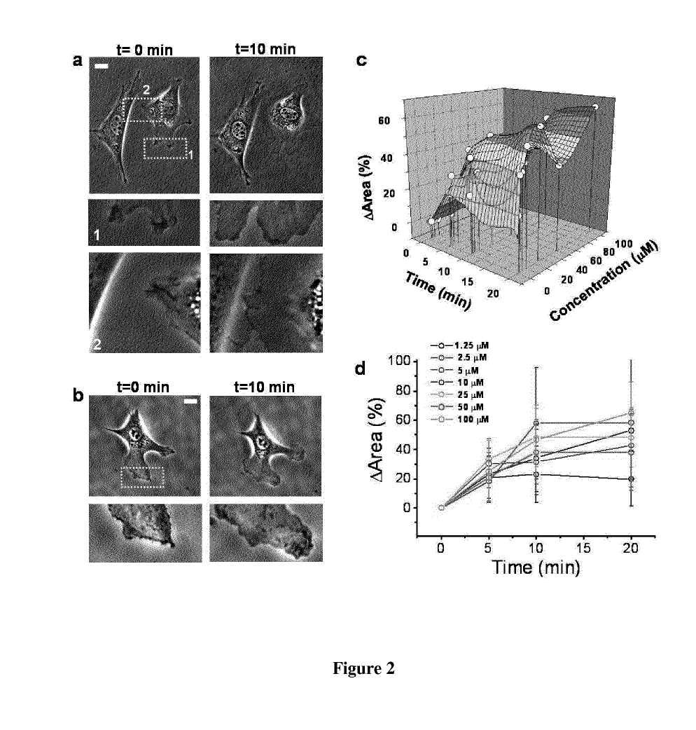

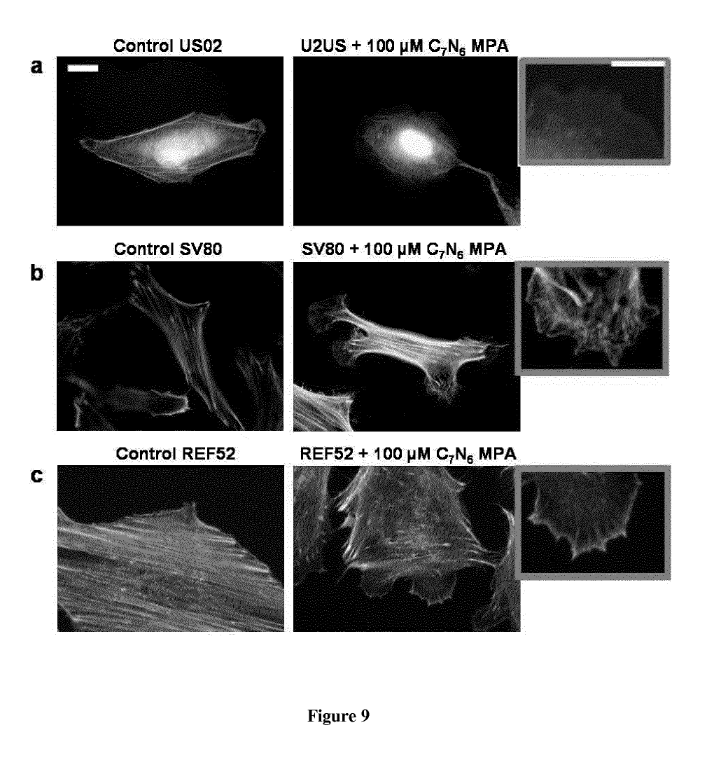

Addition of any one of the 4 compounds to the cell culture medium induced formation of flat protrusions with an unprecedented efficiency and speed. The effect was observed on 4 different cell types (see FIG. 2 and FIG. 9). FIG. 2 shows the responses of cells in the presence of branched C₈N₆ BPA (FIG. 2a) and cyclic C₇N₆ MPA (FIG. 2b). In both cases, within 10 minutes, protrusions were observed extending into sheets morphologically similar to classical lamellipodia. The effect was more pronounced on the edges of cells already exhibiting a lamellipodial activity before the addition of the drugs. However, ectopic lamellipodia were also observed to grow at other locations. As a secondary effect of cells spreading out, neighbouring cells were able to establish contacts de novo (see FIG. 2a, zoomed region 2).

The efficiency of each compound in promoting the growth of lamellipodia was evaluated by measuring the increase in cell areas at several concentrations and different time intervals (see Methods). Optimal values for each circumstance were derived from the resulting 3D graph (FIG. 2c) and the projected graphs (FIG. 2d and FIG. 10) for C₈N₆ BPA. This way of quantifying the effect yields estimates for the efficiency. Note that lamellipodia growth can be modulated by non-linear effects associated with fluctuations in protein concentrations or in feedbacks in signaling pathways. These measurements thus provide rough indications of the conditions for optimal effects rather than a detailed analysis of the concentration dependence of the effects of the drugs.

As expected, the maximal increase was reached faster (within 5 min) at higher concentrations. The different compounds exhibited different behaviour in time response and in maximal increase in area (see FIG. 10a-h). These data suggest that both compounds C₈N₆ BPA and C₇N₆ MPA engender similar efficiencies for growth, but at lower concentrations for C₈N₆ BPA. This also suggests that the distance set by the length of the chain between the two terminal triamino subunits, rather than the molecular topology, determines the efficiency of the drug. The following assays have been carried out with all compounds and similar results were obtained. For simplicity only results obtained with C₈N₆ BPA and C₇N₆ MPA are shown below. Note that the Inventors used 100 μM concentration in most of the experiments in order to promote a dramatic and saturating effect for the growth of lamellipodia; however lower concentrations such as 10 μM worked almost as well.

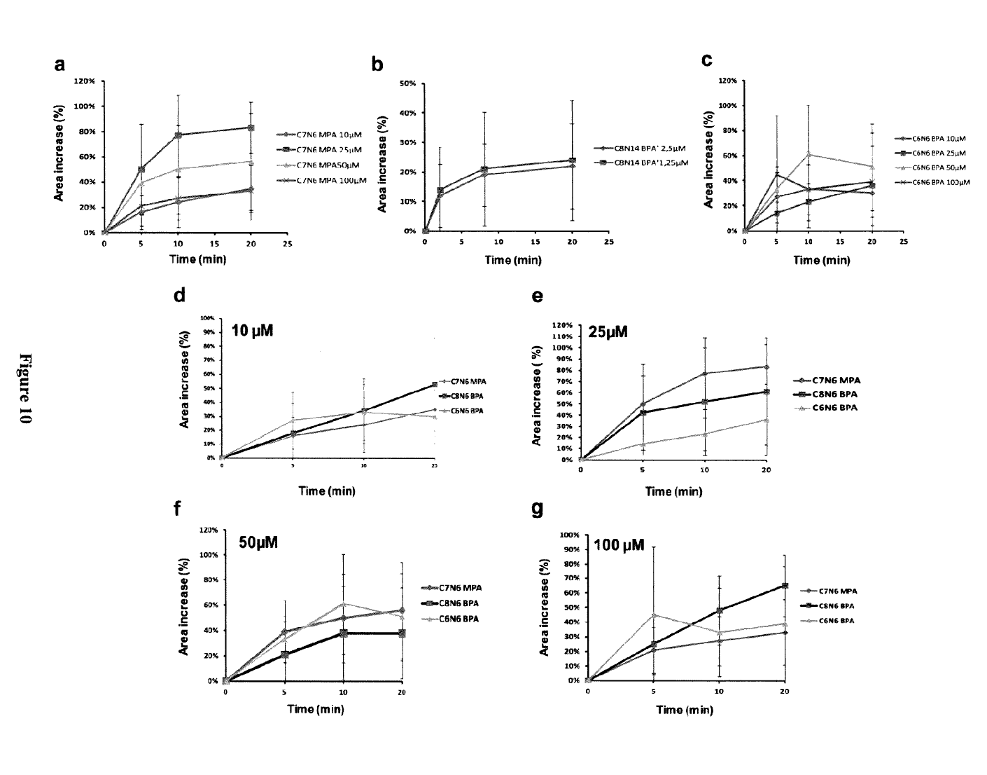

To test the effect of polyamine (C₇N₆ MPA) treatment on cell migration, control (untreated) and treated SW480 cells were seeded in a Boyden chamber with or without C₇N₆ MPA 100 μM, and migration was assessed 24 h later. As shown in FIG. 11a, treatment with the actin-interacting agent could significantly inhibit the migration properties of SW480 cells compared with the control cells (32+/−2 cells/field vs 62+/−12 cells/field, Student t-test, P=0.03). The Inventors then evaluated if this effect was dependent on a reduced cell viability and analyzed the SW480 cell survival fraction by trypan blue exclusion after 24 h and 48 h exposure to increasing concentrations of superpolyamine. These data clearly demonstrated that colon cancer cell line survival was not significantly modified even at the highest concentration (1 mM) (FIG. 11b), suggesting that the effects of the polyamine on cell migration were not associated with cell death.

Cell Entry and Growth Promotion of Lamellipodia

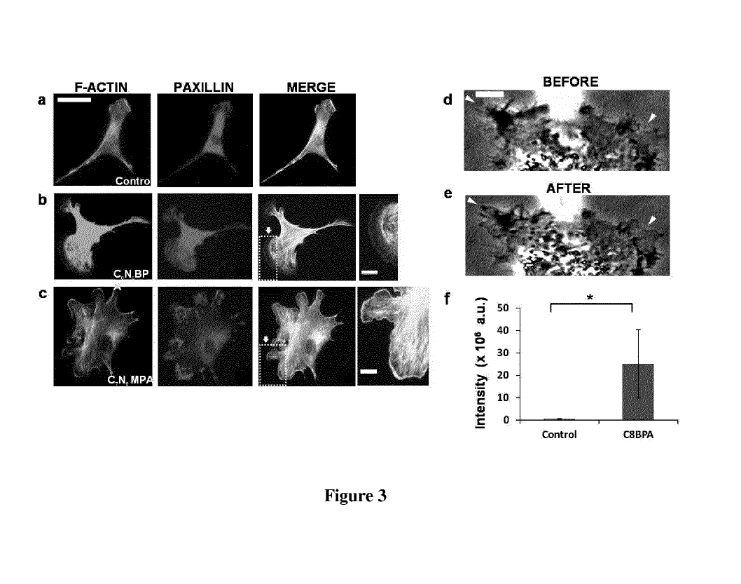

Upon addition of the compounds, cell interaction with the extracellular matrix could be impaired, causing the formation of cell extensions that could differ from the canonical lamellipodia. To test this hypothesis, immunofluorescence localization of focal contact protein paxillin was performed on cells treated with C₈N₆ BPA and C₇N₆ MPA: cells exhibited the expected distribution and shapes of focal contacts⁷, which suggest that the drugs are promoting the growth of normal lamellipodia (see FIG. 3a-c).

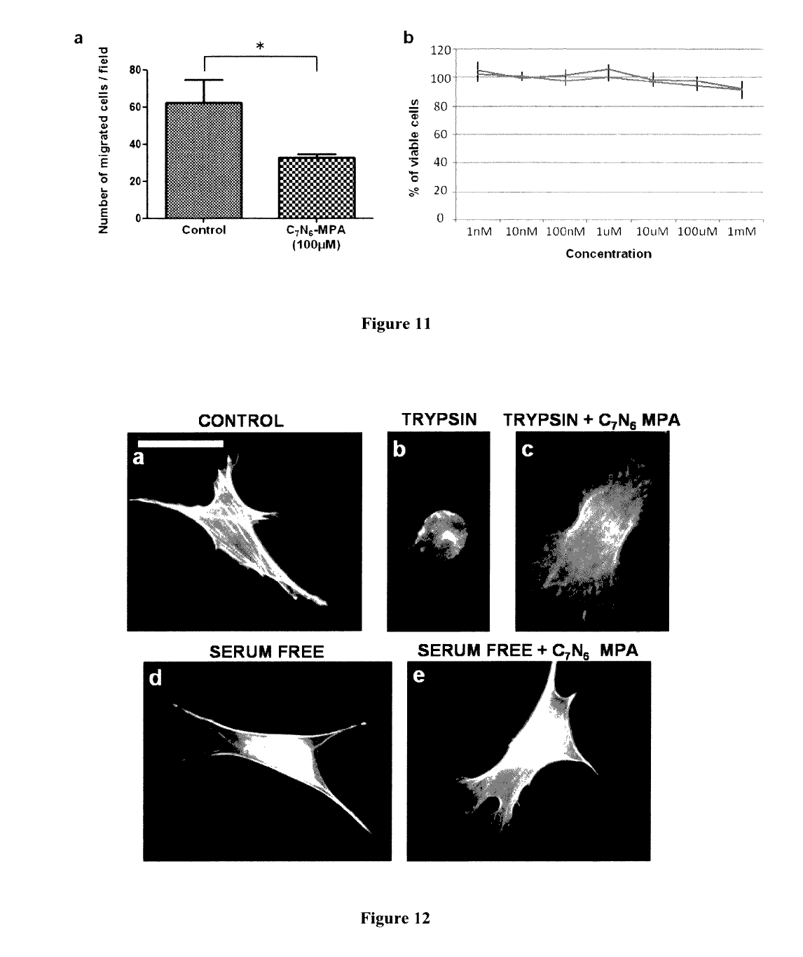

However, the pre-existence of cell focal contacts was not required for drug-promoted growth of cell extensions. Cells were gently detached and forced to round by incubation with a low concentration of trypsin (see FIG. 12a-c); after washing and addition of C₇N₆ MPA, lamellipodia were promoted within 10 minutes, whereas control cells did not re-spread within this time period. This result demonstrates that the lamellipodium grows with de novo formation of focal contacts.

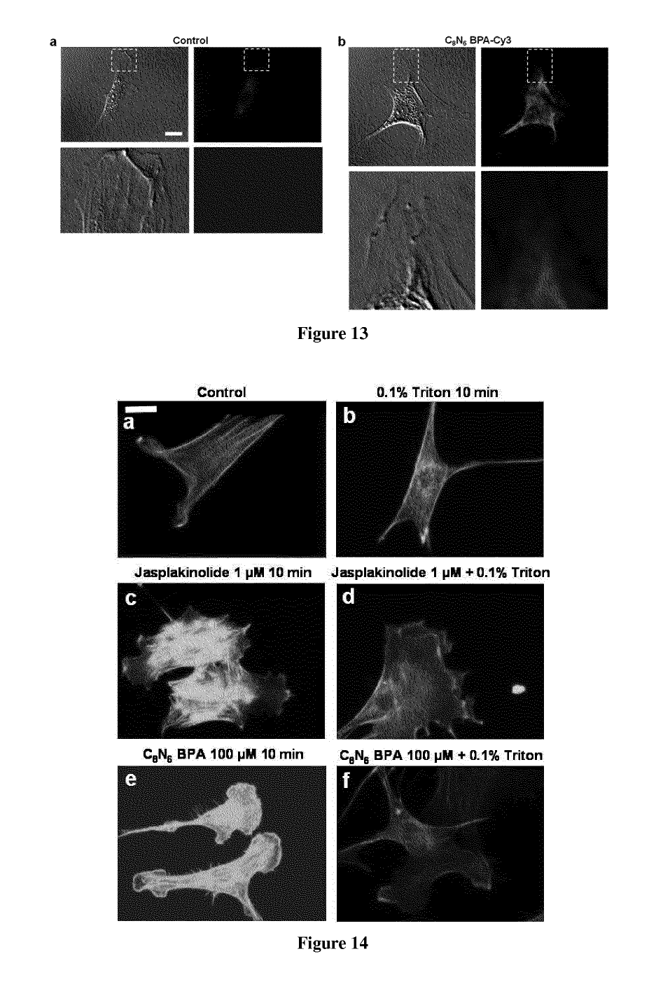

The drugs could be acting on the actin cytoskeleton through an indirect mechanism, for instance by activating some upstream signaling molecule or by modifying the organization of the plasma membrane. Systematic checks were run to see that this was not the case. Repetition of the experiments in the absence of serum (see FIG. 12d-e) showed that within 10 minutes, lamellipodia grew to the same extent as in the presence of serum. Further, injection of the compounds into the cells resulted in stimulation of lamellipodia in an identical manner, demonstrating that: (i) indirect effects on the membrane were not involved; (ii) the compounds entered the cell (FIG. 3d-e); and (iii) their effects simply resulted from their cytosolic localization. This last point was confirmed using 100 μM Cy3-labeled C₈N₆ BPA (see FIG. 3f and FIG. 13) which entered the cell and displayed the same effect as the unlabeled C₈N₆ BPA.

Taken together, these data suggest that the compounds penetrate the cells and promote growth of lamellipodia by targeting actin directly without the need for serum co-factors.

Even though the uptake seems to be immediate, the compounds could be entering by passive diffusion or by carrier-mediated transport, as has been shown for polyamine and polyamine derivatives^{8,9}.

Strikingly, bundles of actin arranged in microspikes were observed within the lamellipodial array when C₈N₆ BPA was added (see FIG. 3b, white arrow, right panel). This feature is consistent with earlier observations of actin networks in lamellipodia¹⁰. The Inventors therefore sought to prove the potential bundling of actin filaments by polyamines in vitro.

Promotion of Bundling of Actin In Vitro by Polyamines

Macrocyclic polyamines have earlier been shown to induce assembly of actin into large aggregates in a low ionic strength buffer in which actin itself remains monomeric⁵. These aggregates were thought to be similar to those induced by polycations like spermine and spermidine¹¹⁻¹³, poly-L-lysine, histones, polysaccharides and charged phospholipids; however, the macrocyclic polyamines appeared much more efficient in promoting actin aggregation than the large charged polyelectrolytes. The aggregates induced by cyclic polyamines were shown to be dissociated by ATP, which interacted with cyclic polyamines in competition with actin⁵.

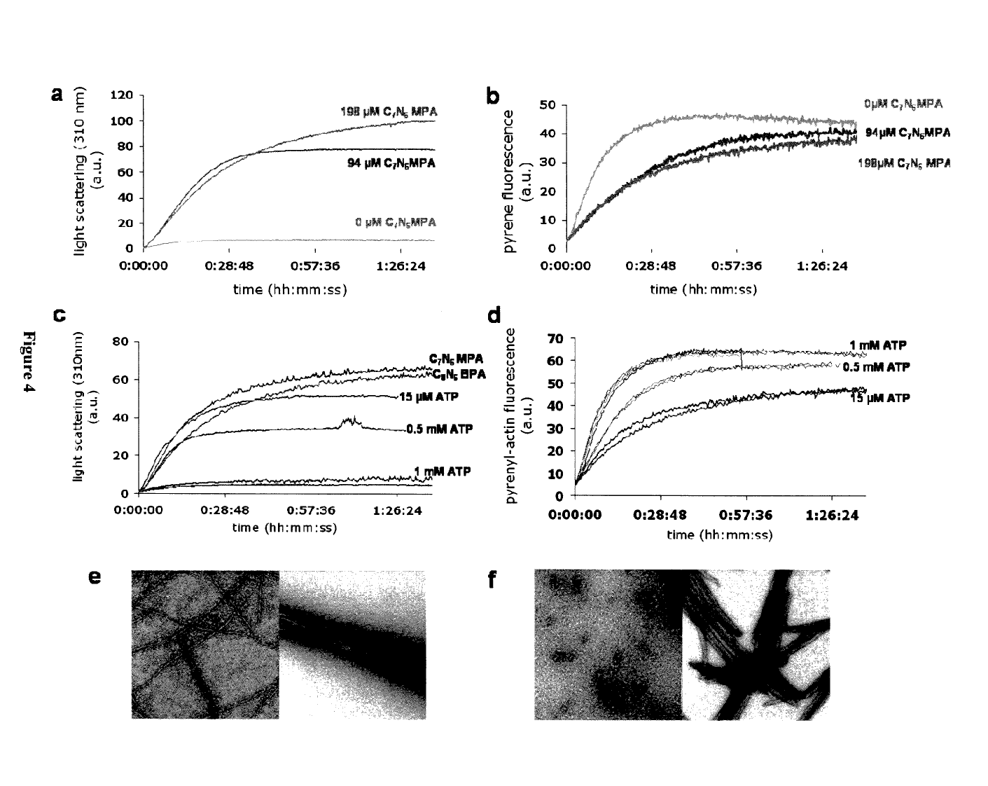

The Inventors checked that the compounds were not changing the pH in vitro in the weakly buffered solution, even at 300 μM. At physiological ionic strength (1 mM MgCl₂, 0.1 M KCl), cyclic polyamines (C₇N₆ MPA) as well as branched polyamines (C₈N₆ BPA) induced assembly of actin (Supplementary Methods), although less efficiently than at low ionic strength. The assembly of G-actin in bundles of filaments was observed in a range of micromolar amounts of actin and 10 to 300 μM polyamines. It was characterized by the associated simultaneous increases in light scattering and in pyrenyl-actin fluorescence (FIG. 4a-b), and by electron-microscopy observation of negatively stained samples (FIG. 4e-f). Finally, the Inventors observed that ATP inhibited the formation of bundles in a dose-dependent manner (FIG. 4c-d).

These results show that the bundling of actin occurs both in vitro and in vivo. The Inventors next probed the specificity of the compounds in vivo by using inhibitors.

Lamellipodia Growth Without Cell Contractility

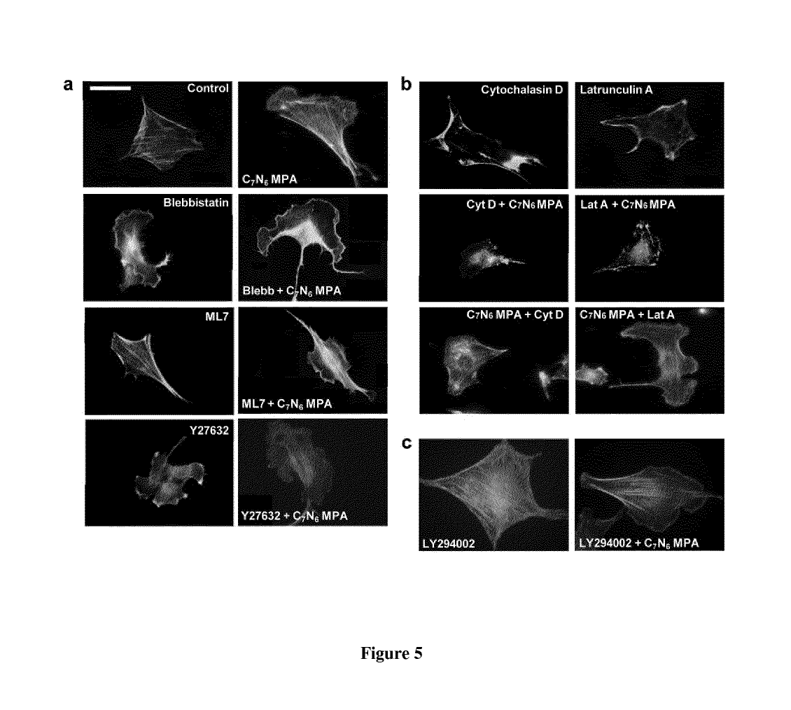

When acto-myosin interaction was inhibited by blebbistatin or by ML-7, the lamellipodia were still growing after addition of the compounds (FIG. 5a). Growth was also observed when Rho kinase was inhibited by the classical Y27632 drug (FIG. 5a bottom).

Inactivity of Polyamine in the Presence of Actin Inhibitors

In contrast, the polyamine compounds had no effect when cellular actin polymerization was prevented by prior incubation with the depolymerizing drugs Cytochalasin D or Latrunculin A (FIG. 5b). This suggests that a key target is indeed actin and that the ability of actin to self-assemble is required for the drug to cause lamellipodia extension.

The Inventors next examined the effect of the compounds on well dispersed cells on surfaces coated with high density of fibronectin. Growth of lamellipodia was not recorded; when plated on densely coated surfaces, cells were extensively spread and showed large lamellipodia. This phenomenon was associated with extensive F-actin assembly by the cells, thus depleting the G-actin source available in standard conditions. Not surprisingly, the compounds did not promote further extension of the lamellipodia in this situation, probably because the cells were lacking a source of available G-actin.

The Inventors further examined how the dynamics and stability of actin arrays pre-assembled by polyamines were affected by the depolymerizing drugs Cytochalasin D and Latrunculin A (FIG. 5b). While addition of Cytochalasin D (1 μM) to C₇N₆ MPA-pretreated cells caused disruption of actin arrays, the lamellipodia induced by C₇N₆ MPA remained assembled upon addition of Latrunculin A (1.5 μM). The failure of the lamellipodial actin arrays to disassemble upon addition of the actin sequestering drug suggests that these arrays are unusually stable. Consistently, when cells were permeabilized by Triton¹ the lamellipodial arrays induced by polyamines resembled jasplakinolide-treated cytoskeletons (FIG. 14).

The Inventors next studied the interaction between the compounds and acidic lipids. These compounds could bind lipids, thereby activating the PI3 kinase associated with lamellipodial growth. The Inventors observed that no growth was promoted when the PI3 kinase inhibitor (LY294002)¹⁴ was added (FIG. 5c). However, the Inventors saw lamellipodial growth when the compounds were subsequently added. This confirms the direct effect of the compounds on actin.

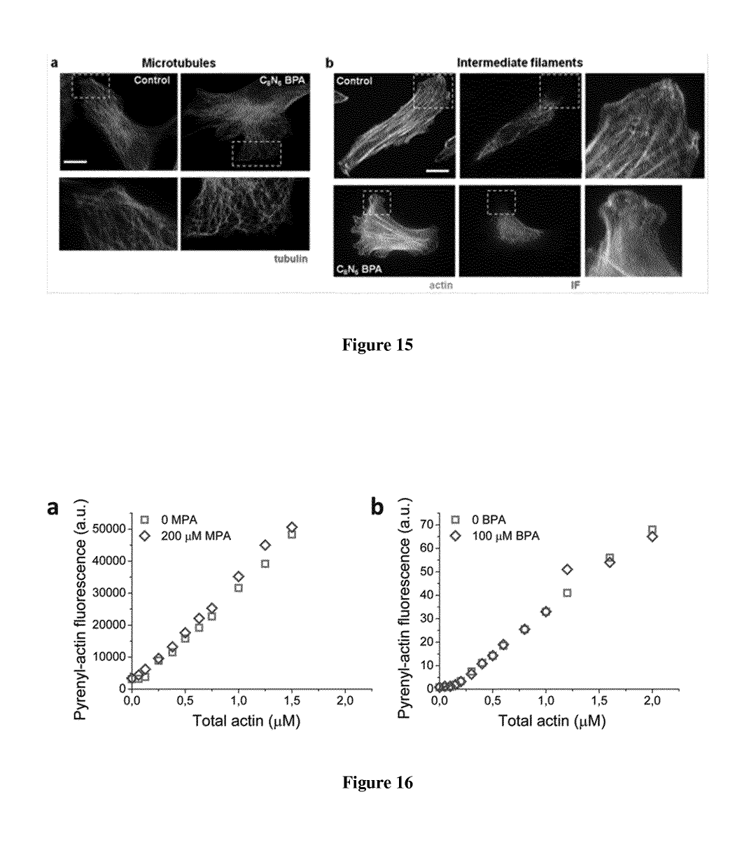

Positively charged molecules were shown to induce the bundling of cytoskeletal polymers by neutralizing charges. The Inventors therefore checked whether the compounds were altering the organization of microtubules and intermediate filaments: no difference was observed in their organization (see FIG. 15). This further shows the specificity of these synthetic polyamines.

Altogether these results show that the in vivo effects of the polyamine drugs result from their functional interaction with regulated actin assembly leading to lamellipodial arrays. The Inventors therefore sought to characterize their effects on actin assembly dynamics in vitro using bulk solution assays and reconstituted motility assays.

Specific Retardation of Barbed-End Growth by Polyamines

Preliminary polymerization assays performed using either light scattering or pyrenyl-actin fluorescence as probes indicated that spontaneous assembly at physiological ionic strength is slowed down by polyamines (FIG. 4b). Whether polyamines inhibit nucleation or elongation of actin filaments cannot be discerned by the spontaneous assembly assay.

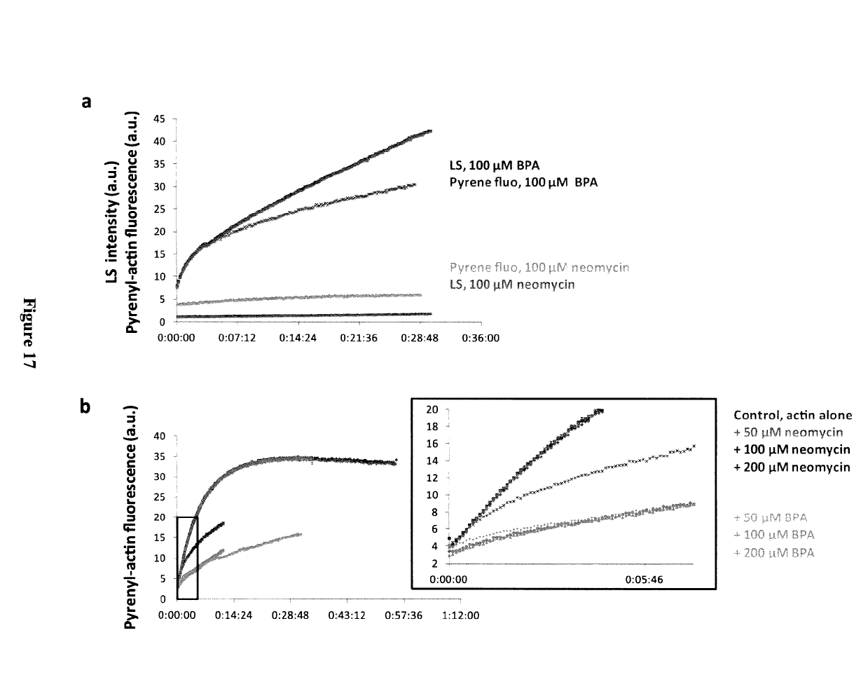

Seeded filament growth assays therefore were performed using spectrin-actin seeds and gelsolin-actin complexes, which initiate barbed-end and pointed-end growth of filaments respectively. FIG. 6a shows that barbed-end growth was specifically inhibited by about 85% by MPA in the range 0 to 150 μM. Pointed-end growth, in contrast, was only slightly decreased in the same concentration range. On the other hand, when barbed-end growth was initiated by the FH₁-FH₂ construct of formin mDial, the half-inhibition of barbed-end assembly by C₇N₆ MPA required a three-fold higher amount of cyclic polyamine (FIG. 6Ab), indicating that formin binding to barbed ends somehow protects them from the inhibition by polyamines. Dilution-induced depolymerization assays further showed that barbed-end depolymerization was slowed down to the same extent as barbed-end growth by C₇N₆ MPA (FIG. 6Ac). Although the kinetics of barbed-end assembly and disassembly were dramatically slowed down by C₇N₆ MPA, the critical concentration for filament assembly appeared unaffected in measurements of F-actin assembled at steady state (FIG. 16a). Similar results were obtained with branched polyamines (FIG. 16Ab).

Since the Inventors showed that bundling was promoted by the compounds, the Inventors evaluated whether bundling was responsible for the assembly kinetics. Bundling of filaments by polyamines is less prominent at physiological ionic strength than at low ionic strength, and shows a higher concentration dependence range than the range at which extension of lamellipodia and slower barbed-end kinetics are observed. Although the involvement of bundling in the kinetic effects of superpolyamines cannot be completely discarded, the fact that the thermodynamic stability (critical concentrations at barbed and pointed ends) of the actin arrays do not appear modified suggests that the effects differ from those observed with bundling agents that bind in a 1:1 molar ratio to actin along the sides of filaments and stabilize actin arrays, like Eps8¹⁵ or EF1alpha¹⁶.

In conclusion, polyamines do not affect the stability of filaments but slow down their assembly-disassembly dynamics. The electrostatic nature of the effect of polyamines on barbed-end growth was demonstrated by performing seeded growth assays at various ionic strengths in the absence and presence of 100 μM C₇N₆ MPA and in a polymerization buffer containing 1 mM MgCl₂ and increasing KCl in the range 0 to 200 mM. The percent inhibition of barbed-end growth decreased upon increasing ionic strength and was undetectable above 150 mM KCl (FIG. 6Ad).

Retardation of Treadmilling by Polyamines

Actin-based motile processes result from site-directed initiation of actin assembly by two major signal-responsive protein machineries. WASP family proteins use the Arp2/3 complex to branch filaments at the membrane and assemble a dendritic protrusive meshwork, while formins initiate barbed-end processive assembly of actin bundles. In both cases, force is produced by barbed-end growth fueled by regulated treadmilling. Treadmilling that drives the formation of dendritic actin arrays is enhanced by the synergistic effects of capping proteins, profilin and Actin Depolymerizing Factor (ADF/cofilin). ADF is known to destabilize actin filaments, promoting a large increase in pointed end depolymerization rate, the limiting step in filament treadmilling¹⁷. FIG. 6 Ba-c confirms that gelsolin-capped filaments depolymerize up to a maximum of 30-fold faster in the presence of ADF, in agreement with previous data¹⁷. The gelsolin-capped filaments are too short (1 μm in length) to be severed by ADF, since the average length of ADF-decorated filaments is 3 μm, only 3-fold shorter than the average length of standard actin filaments¹⁸, hence the measured effect is only due to an increase in depolymerization rate constant. In the presence of cyclic or branched polyamines, the rate of depolymerization was much less enhanced by ADF (about three-fold at 3 μM ADF, versus 30-fold in the control without polyamines). However, a sedimentation assay showed that ADF caused the same partial depolymerization and bound F-actin similarly in all samples with and without C₇N₆ MPA or C₈N₆ BPA (FIG. 6Bc). In other words, the steady state of actin assembly in the presence of ADF is practically unaffected by C₇N₆ MPA or C₈N₆ BPA. Finally, note that other bundling factors like fascin did not affect the kinetics of F-actin depolymerization by ADF in vitro.

In conclusion, in this assay again cyclic and branched polyamines slow down the dynamics of actin filaments and abolish the effect of ADF, but do not affect the thermodynamics of actin binding to ADF.

Retardation of Propulsion of N-WASP-Coated Beads

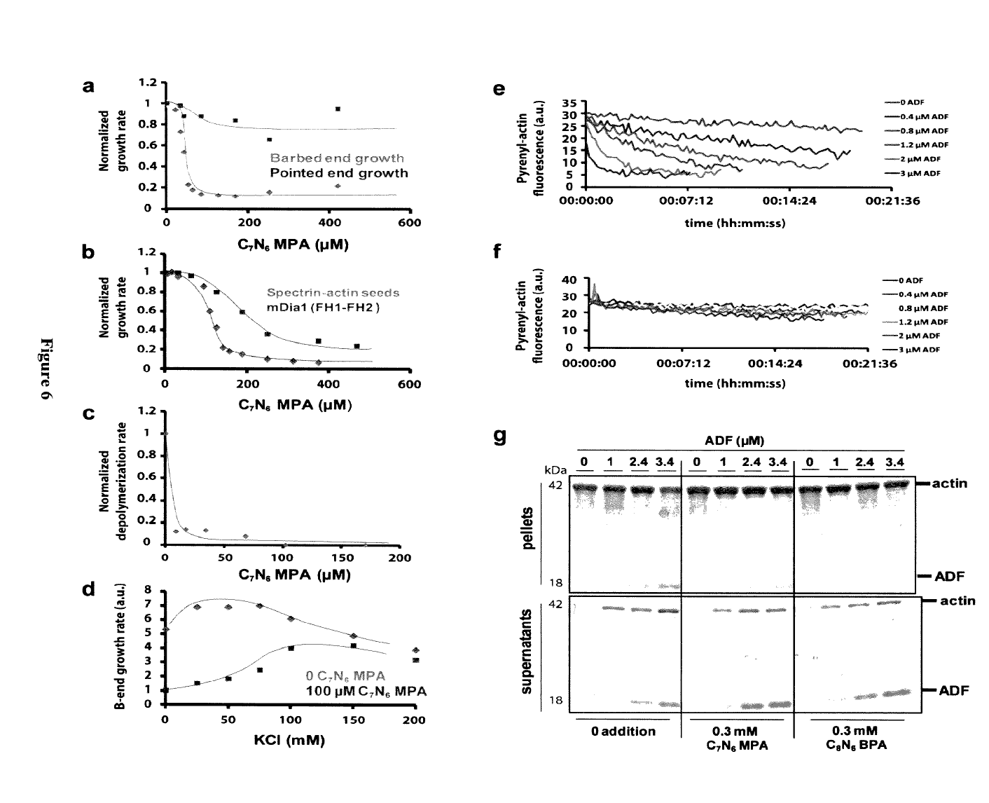

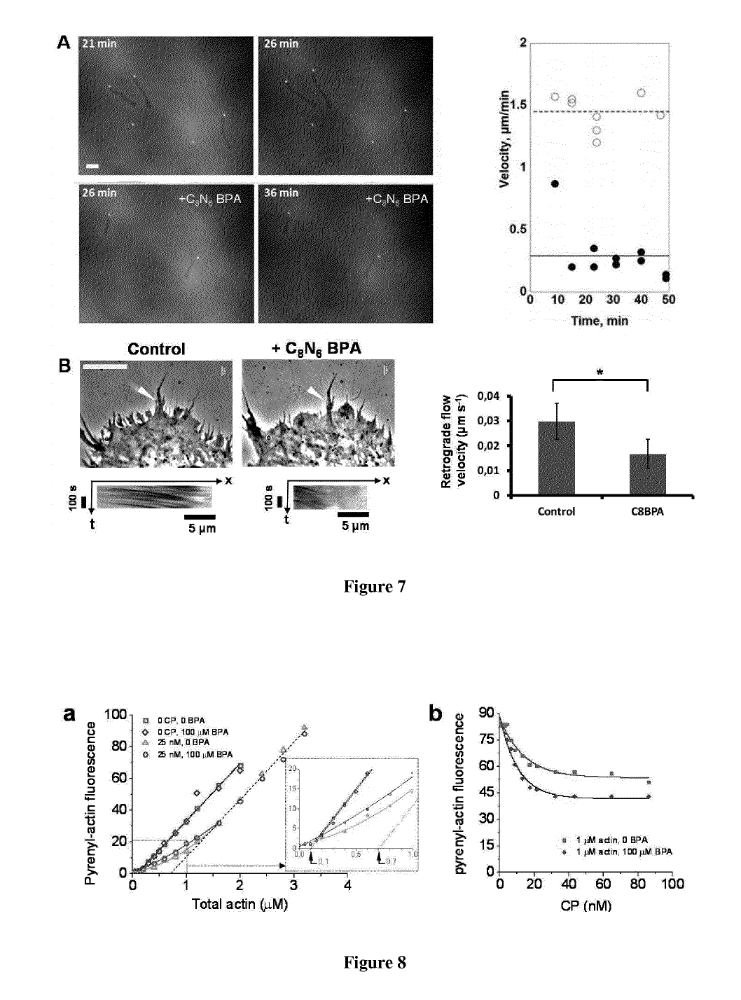

In parallel to these assays, the Inventors examined the effect of C₈N₆ BPA on the propulsive movement of N-WASP-coated beads in a reconstituted motility assay containing F-actin, Arp2/3, ADF, profilin and gelsolin¹⁹. Addition of 50 μM C₈N₆ BPA a few minutes after initiation of comet tail formation caused a dramatic decrease in propulsion rate (FIG. 7a).

The Inventors have shown that the polyamine compounds slow down the treadmilling of actin filaments in vitro. To verify that the turnover of lamellipodial actin arrays induced by the drugs is slowed down also in vivo, the Inventors complemented these experiments by measuring the retrograde flow (see FIG. 7b) allowing proper visualization of the flow²⁰ (see Methods). Results showed that this flow is decreased in the presence of the polyamines. Altogether, these data show that in vivo as well, actin turnover is slower in the presence of the polyamine compounds. This leads to the stabilization of the lamellipodial branched array.

Promotion of Enhanced Actin Nucleation In Vitro

While the above data explain why the lamellipodia were stabilized by the polyamines, in vitro data do not explain how lamellipodia could be initiated by the drugs. To address this point, the Inventors performed the following experiments.

The lamellipodial branched array requires the combined actions of filament branching directed at lamellipodium tip by WAVE protein using the Arp2/3 complex, and other regulators like ADF and capping proteins that enhance treadmilling. The Inventors had measured that the effect of ADF was weakened by polyamines. The effect of polyamines on capping protein activity was evaluated next.

Capping proteins are required to maintain an environment in which most barbed ends are capped, thus establishing a high steady state amount of G-actin, close to Cc of pointed ends. In addition, capping proteins block barbed-end growth of filaments nucleated by branching, thus regulating their length (architecture of the network) and lifetime. An optimum concentration range of capping protein is defined for maximum propulsion rate²¹, above which bead velocity decreases, because filament growth is arrested immediately after branching and nucleation of the daughter filament has occurred, thus generating a very densely branched and slowly growing network¹⁹.

In solutions of pure actin, in the absence of capping proteins, critical concentration plots consist of two straight lines, indicating that a sharp transition exists at the critical concentration between the monomer and the polymer states. In contrast, barbed-end capping generates critical concentration plots that show a curvature in the region of the critical concentration, indicating that below and slightly above the extrapolated critical concentration, short polymers are stabilized by the capping protein, as a result of the lowered free energy of nucleation. Most barbed-end cappers including Capping Protein and gelsolin actually nucleate actin. While in the absence of capping protein the critical concentration plots obtained in the absence or presence of BPA are strictly superimposable, in the presence of capping protein they superimpose at high actin concentration but display a different curvature in the region of the critical concentration when BPA is present. The same effect was seen when the Inventors measured the dependence of the amount of F-actin at steady state on the concentration of added capping protein, at 1 μM total actin and in the presence of BPA/MPA (FIG. 8). The data points are all above the ones observed in the control plot of data obtained in absence of BPA, indicating that BPA stabilizes a greater number of short polymers in the presence of capping protein.

The free energy of filament nucleation thus appears lowered to a larger extent with BPA than without BPA. The capping protein is in slow association-dissociation equilibrium with barbed ends. The Inventors propose that the fraction of oligomers that is not bound to capping protein at any time can therefore interact with a nucleating factor and become a stabilized growing filament.

In other words, in the presence of capping protein, BPA would maintain a larger reservoir of potential actin filament pre-nuclei, thus facilitating filament branching by WAVE-Arp2/3.

These results lead to the suggestion that polyamines enhance nucleation by capping protein, thus facilitating the initiation of filament branching at the lamellipodium tip.

Discussion

The present results bring mechanistic insight into two different aspects of actin-based motility. First, the Inventors provide evidence that small branched or cyclic polyamines promote growth of lamellipodia in various cell types; second, the effect of polyamines appears to be mediated by their regulation of actin nucleation and turnover in the cellular context, which sheds light on the potential mechanisms used by other cellular regulators of motility. These results offer the prospect of potential applications of polyamines as actin-specific tools in cell dynamics and medicine. The Inventors briefly review these perspectives below.

Polyamines are cell-permeant and their effects in vivo reproduce the effects measured in vitro on simple systems composed of actin and regulators of treadmilling such as ADF and Capping Protein. These small size polyamines act in two complementary ways: they slow down the turn-over of actin within the existing lamellipodia and they enhance nucleation of actin at the cell edge.

All polyamines like poly-L-lysine, protamines, and histones are basic compounds, known to cause bundling of polyelectrolytes like actin filaments^{22,23} by neutralizing negatively charged surface residues, e.g. the exposed aspartate and glutamate residues of the N-terminus of actin²⁴. Small branched or cyclic polyamines (BPA/MPA) follow this rule and cause bundling of filaments in an ionic strength sensitive fashion; however they display additional particular properties that are responsible for their specific effects on actin dynamics. The data showing a high affinity inhibition of barbed-end assembly-disassembly kinetics suggest that binding of these polyamines to the terminal F-actin subunits alters the structure of the barbed end specifically. Further experimentation using microfluidics allied to TIRF microscopy to study individual filament dynamics, as well as more detailed structural molecular dynamics approaches, will be essential to evaluate this possibility. Consistent with this possibility is the narrow range of distances between active groups on polyamines in which the compounds display their regulatory activity on actin dynamics.

Other amino-compounds like neomycin have been reported to induce lamellipodia (for example, Distribution of cytoskeletal proteins in neomycin induced protrusions of human fibroblasts²⁵), most likely by acting on surface receptors. The Inventors have verified that up to at least 100 μM, neomycin does not induce bundling of actin at low ionic strength in vitro, while massive bundling is induced by BPA at that concentration, and does not inhibit barbed-end growth either (FIG. 17). Hence the effects of neomycin on cell motility are unlikely to be mediated by direct interaction with actin as those observed here with branched and cyclic superpolyamines.

Remarkably, lamellipodia rather than filopodia appear to be induced by BPA/MPA. This observation, which is counterintuitive at face value, since filopodia are made of bundles and might be expected to be favored by polyamines, is actually fully consistent with the in vitro observation that binding of formins (which promote filopodia) to barbed ends and successive assembly eliminate the effect of BPA/MPA on barbed-end dynamics (FIG. 6Aa-d).

The slower assembly-disassembly dynamics of pure actin in vitro, and the resulting decreased velocity of N-WASP-coated beads propelled by site-directed assembly of a Arp2/3-branched actin meshwork may have implications in vivo^{26,27}. In particular, the Inventors found that cell migration was slowed down in the assay with Boyden chambers. This is expected since motion is supported by barbed-end growth of WAVE-stimulated barbed-end assembly of an Arp2/3-branched actin array. Slower migration was actually measured in a variety of cell types following up to 24 hours treatment by polyamines. It is possible however that slower migration results in part from the effect of polyamines on actin turnover at adhesions, which is coordinated to actin turnover in protrusive structures. Along the same line, polyamines may slow down other actin-based processes dependent on regulated treadmilling, such as re-organization of the Golgi apparatus²⁸, scission of tubulated membranes by WASH/Arp2/3²⁹, dendritic spine dynamics in synaptic plasticity³⁰ and the dynamics of immune synapse formed in T-cell activation^{31,32}. BPA/MPA may play an instrumental role in the kinetic analysis of other processes such as cytokinetic ring closure, in which ADF-mediated disassembly of actin filaments contributes to contractility^{33,34}. Similarly, analysis of morphogenetic processes characterized by finely tuned rapid dynamics of actin filaments, such as the establishment of oocyte polarity in Drosophila³⁵ or spindle translocation in asymmetric meiotic division, may benefit from the use of BPA/MPA. Actually, in slowing down actin dynamics to desired levels, polyamines prove more manageable tools than Latrunculin A or Cytochalasin D to identify the key elementary steps.

Polyamine targeting of the actin cytoskeleton represents an alternative to the use of specific reagents to block various cell functions. Polyamines are abundant in cells and play an important role in normal metabolism. Choosing those synthetic polyamines that have the appropriate size and geometry to target actin with high affinity will open avenues for manipulating cell motility and cell division and proliferation. This class of compounds will probably have a number of applications both in vitro and in vivo, with strong potential as a tool to study metastasis in cancer.

In conclusion, the Inventors have shown that synthetic molecules, of both macrocyclic and acyclic branched polyamine nature, can modulate actin dynamics in cells by implementing specific supramolecular interactions. These results extend the concept of “superpolyamines” to both classes of compounds. The same approach could be used with other active groups of various geometries and distance between groups. Our work demonstrates that this approach can efficiently target proteins at the nanometre scale in vitro and in vivo. The Inventors anticipate and hope that this new class of compounds will be used in various situations both in vitro and in vivo.

Materials and Methods

Cytoskeletal Drugs

Polyamine compounds were dissolved in Milli-Q water to give a stock solution of 30 mM. For immunostaining and microscopy experiments, polyamine compounds were added to the cell medium at a final concentration of 100 μM, except for experiments which tested efficiency. For immunostaining experiments, NIH3T3 cells were incubated with Blebbistatin (30 μM), ML7 (10 μM), Y27632 (10 μM), Cytochalasin D (1 μM) (Sigma-Aldrich) for 30 min, LY294002 (50 μM) (Sigma-Aldrich) for 30 minutes. Latrunculin A (1.5 μM) (Sigma-Aldrich) was added for 5 min. After additional 20 min incubation with 100 μM polyamine, cells were fixed and stained.

Actin Assembly-Disassembly Assays

Actin assembly was monitored using as probes the increase in light scattering intensity (310 nm, 90° angle) and in the fluorescence of pyrenyl-actin (excitation 366 nm, emission 407 nm). Measurements were made at room temperature in a Xenius Safas spectrofluorimeter. CaATP-G-actin was converted into MgATP-G-actin by addition of 20 μM MgCl₂ and 0.2 mM EGTA to a solution of 10 μM CaATP-G-actin in G buffer. Polymerization was triggered by addition of MgCl₂ (1 mM final), EGTA (0.2 mM final) and KCl as indicated to MgATP-G-actin. The 1:1 CaATP-actin complex was obtained by removing free ATP by two consecutive treatments of CaATP-G-actin in G buffer with 10% suspension vol/vol of Dowex-I (BioRad) in G buffer without ATP.

Seeded barbed end growth assays were performed at 2.5 μM actin and 0.1 nM spectrin-actin seeds³⁸. Pointed end growth assays were performed similarly but using preformed gelsolin-actin complexes (2 nM final) as seeds. The initial rate of fluorescence increase was measured.

Dilution-induced depolymerization assays were performed by 40-fold dilution of a 2.5 μM F-actin (50% pyrenyl-labeled) into F buffer containing BPA/MPA at desired concentrations. The initial rate of fluorescence decrease was measured.

Critical concentration plots were performed by serial dilution of F-actin (10% pyrenyl-labeled) in F buffer containing the desired amounts of BPA/MPA. Pyrenyl-actin fluorescence was read after overnight incubation in the dark.

Reconstituted Motility Assays

Reconstituted motility assays were performed using 2 μm diameter carboxylated polystyrene beads functionalized with N-WASP (170 nM, 2 hours, followed by 1% BSA neutralization for 20 min)¹⁹. Beads were placed in a reconstituted motility mix containing 7.5 μM F-actin, 75 nM Capping Protein, 2 μM profilin, 7 μM ADF, 75 nM Arp2/3 complex in × buffer (10mM HEPES, 0.1M KCl, 1 mM MgCl₂, 0.1 mM CaCl₂, 1 mM ATP, 7 mM DTT, 1.5 mM DABCO at pH 7.8, 0.5% BSA and 0.2% methylcellulose). C₈N₆ BPA was then added at 50 μM. Observations were made using a phase contrast Olympus AX70 microscope controlled by MetaMorph 6.3r7 with CCD camera and 20× phase objective. Data analysis was performed to extract bead velocity with ImageJ software. Bead velocity was measured and averaged on 10-15 beads.

Data Analysis

Increase in cell area for given time point (n=5, 10 and 20 min) was calculated by (A1n-A0)/A0, where A0 and A1n are the areas before and after polyamine addition, respectively. Kymographs for the analysis of actin retrograde flow in the lamellipodia region were performed in ImageJ. Moving features were visualized as diagonal streaks with slope depending on the velocity of movement.

Statistical Analysis

Data is provided as mean±s.d. Statistical analysis was carried out by using two-tailed Student's t-test, and significance was accepted at P<0.05.

Immunostaining

Cells were fixed in 3% PFA (Sigma-Aldrich) for 17 min and then permeabilized with 0.5% Triton solution (Sigma-Aldrich) for 3 min and rinsed twice with 1× PBS. For actin and focal contacts staining, cells were incubated with Alexa 488 phalloidin (1:200, Molecular Probes) and anti-paxillin monoclonal primary antibody (1:50, BD Biosciences) for 45 min at RT. Samples were finally mounted in 50% Glycerol/1× PBS on glass slides and protected from drying. For the intermediate filaments staining, cells were quenched in NaBH₄/PBS solution for 10 min at RT. Cells were incubated first with anti-vimentin (1:500, Convance) for 1 h and then with goat anti-guinea pig Dylight 594 (1:1000, Jackson) for 45 min at RT. Microtubules were fixed using methanol at −20° C. for 10 min and stained with mouse monoclonal anti-alpha tubulin (1:50, Sigma-Aldrich) for 45 min at RT.

Cell Culture and Transfection

NIH3T3 mouse fibroblast and REF52 rat embryo fibroblasts (ATCC) were cultured in DMEM supplemented with 10% calf bovine serum (BCS, Sigma-Aldrich) or fetal bovine serum (FCS, Hyclone) and 1% Pen/Streptomycin (InVitrogen). Cells were grown at 37° C. and 5% CO₂. For microscopy and immunostaining, cells were trypsinized (0.25% Trypsin-EDTA, InVitrogen), centrifuged and plated on glass coverslips. For live cell observation, Leibovitz's L-15 1× medium (InVitrogen) supplemented with 10% serum (FCS for REF52 and BCS for NIH3T3 cells) and 1% antibiotics was used.

For retrograde flow experiments, surfaces were coated with poly-lysine (Sigma-Aldrich) as described in Verkhovsky et al.²⁰.

Proteins

Actin was purified from rabbit skeletal muscle, isolated in CaATP-G-actin form by size exclusion chromatography in G buffer (5 mM Tris-Cl—, pH 7.8, 0.1 mM CaCl₂, 0.2 mM ATP, 1 mM DTT, 0.01% NaN₃) and pyrenyl-labeled as described¹. Human gelsolin, mouse profilin, mouse Capping Protein and human ADF were bacterial recombinant proteins, and Arp2/3 was purified from bovine brain as described in Egile et al.³⁹. His6-tagged human N-WASP was expressed in sf9 cells using the baculovirus expression system and purified as described³⁹. Spectrin-actin seeds were isolated and purified from human blood cells⁴⁰.

Optical Microscopy

Live cell phase-contrast images were acquired with a CKX41 microscope with a 20× and 40× air objectives (Olympus). The microscope was equipped with a cooled CCD camera (CFW-1612M; Scion Corporation) and Micro-Manager software v1.3. Data acquisition frequency was 1 image/30sec with 125 ms of exposure time. The setup was embedded inside a temperature controlled cage at 37° C.

Actin retrograde flow velocity experiments were performed in a Leica DM 2500 Microscope with a 100× oil objective at 37° C. Data acquisition was 1 image/5 sec.

Fluorescent images were taken in a Fluorescence Nikon Ti microscope (60× oil objective, NA 1.25) combined with a Photometrics CoolSNAP HQ2 camera and NIS acquisition software.

Electron Microscopy

Electron microscopy observation of actin assemblies in the presence of C₇N₆ MPA was performed using a Philips CM12 electron microscope at 80 kV. Samples containing actin (2 μM) in either in G or F form and C₇N₆ MPA were prepared and placed on glow-discharged carbon-coated grids.

Migration Assay

SW480 human colorectal cancer cell line was cultured in DMEM supplemented with 10% fetal bovine serum (FBS) at 37° C. All experiments were done at 50% to 70% cell confluence and at early passages and migration assay was conducted in a standard 8-μm-pore Boyden chamber (BD BioCoat, BD Biosciences).

Briefly, SW480 cells were starved for 4 h and equal numbers (50.000 cells per well) of control or polyamine (C₇N₆)-treated (100 μM) cells were suspended in 500 μL of DMEM—1% FBS with or without C₇N₆MPA polyamine and placed in the top compartment of the chamber. 750 μL of DMEM—10% FBS with or without superpolyamine was added to the bottom compartment. Following 24 h incubation, non-migrating cells were carefully removed from the top compartment with a cotton swab and cells that had migrated to the bottom compartment were fixed in 4% PFA and stained with DAPI. Cells were washed three times in 1× PBS and mounted using Vectashield mounting medium (Vector Laboratories). Fluorescence images were captured using an Olympus AX60 epifluorescent microscope equipped with a CCD camera and analyzed with AnalySIS software (Soft Imaging System). The number of migrated cells was determined from ten random fields visualized at ×200 magnification.

Cell Viability Assay.

To determine the effect of superpolyamine (C₇N₆ MPA) (1 nM-1 mM) on SW480 cell line survival, cells were seeded at a density of 3.10⁴ per well (500 μL) in 24-well flat-bottomed plates and viability was determined 24 h or 48 h after exposure to the drug by cell counting and trypan blue dye exclusion. Cells collected by trypsinization were stained with trypan blue and the viable cells in each well were counted. The viability of the untreated cells was regarded as 100%.

### REFERENCES

1. Cramer, L. P. Curr. Biol. 9, 1095-1105 (1999).

2. Peterson, J. R. et al. Nat. Struct. Mol. Biol. 11, 747-755 (2004).

3. Nolen, B. J. et al. Nature 460, 1031-1034 (2009).

4. Rizvi, S. A. et al. Chem. Biol. 16, 1158-1168 (2009).

5. Oriol-Audit, C., Hosseini, M.W. & Lehn, J.-M. Eur. J. Biochem. 151, 557-559 (1985).

6. Mita, N. et al. EP1840127 A1 (Ciba Sc Holding, Japan, 2007).

7. Deakin, N. O., Ballestrem, C. & Turner, C. E. PLoS ONE 7, e37990 (2012).

8. Casero, R. A. & Marton, L. J. Nat Rev Drug Discov 6, 373-390 (2007).

9. Soulet, D., et al., J Biol Chem 279, 49355-49366 (2004).

10. Svitkina, T. M. et al. J. Cell. Biol. 160, 409-421 (2003).

11. Oriol-Audit, C. Eur. J. Biochem. 87, 371-376 (1978).

12. Oriol-Audit, C. Biochem. Biophys. Res. Commun. 105, 1096-1101 (1982).

13. Grant, N., Oriol-Audit, C. & Dickens, M Eur. J. Cell Biol. 30, 67-73 (1983).

14. Vlahos, C. J., et al. J. Biol. Chem. 269, 5241-5248 (1994).

15. Hertzog, M. et al. PLoS Biol 8, e1000387 (2010).

16. Murray, J. W., et al. J. Cell Biol. 135, 1309-1321 (1996).

17. Carlier, M. F. & Pantaloni, D. J. Mol. Biol. 269, 459-467 (1997).

18. Pfaendtner, J., De La Cruz, E. M. & Voth, G. A. A. Proc. Natl. Acad. Sci. USA. 107, 7299-7304 (2010).

19. Wiesner, S. et al. J. Cell Biol. 160, 387-398 (2003).

20. Verkhovsky, A. B. et al. Mol. Biol. Cell. 14, 4667-4675 (2003).

21. Loisel, T. P., et al., M.-F. Nature 401, 613-616 (1999).

22. Tang, J. X. & Janmey, P. A. J. Biol. Chem. 271, 8556-8563 (1996).

23. Tang, J. X., et al. Biochemistry 36, 12600-12607 (1997).

24. Fujii, T., Iwane, A. H., Yanagida, T. & Namba, K. Nature 467, 724-728 (2010).

25. Safiejko-Mroczka, B. & Bell Jr, P. B. Exp. Cell Res. 242, 495-514 (1998).

26. Suraneni, P. et al. J. Cell Biol. 197, 239-251 (2012).

27. Taylor, M. J., Lampe, M. & Merrifield, C. J. PLoS Biol. 10, e1001302 (2012).

28. Campellone, K. G., et al. Cell 134, 148-161 (2008).

29. Derivery, E. et al. Dev. Cell. 17, 712-723 (2009).

30. Hotulainen, P. et al. J. Cell Biol. 185, 323-339 (2009).

31. Huang, Y. & Burkhardt, J. K. J. Cell Sci. 120, 723-730 (2007).

32. Jia, D. et al. W Proc. Natl. Acad. Sci. USA 107, 10442-10447 (2010).

33. Mendes Pinto, I., et al. Dev. Cell 22, 1247-1260 (2012).

34. Wiggan, O. N., et al. Dev. Cell 22, 530-543 (2012).

35. Dahlgaard, K., et al. Dev. Cell. 13, 539-553 (2007).

36. Jardim, M. G., et al. J. Eur. J. Inorg. Chem. 2010, 1729-1735 (2010).

37. Hosseini, M. W. & Lehn, J.-M. Helv. Chim. Acta 69, 587-603 (1986).

38. Husson, C., et al. Cordon-Bleu uses Mol. Cell. 43, 464-477 (2011).

39. Egile, C. et al. J. Cell Biol. 146, 1319-1332 (1999).

40. Casella, J. F., Maack, D. J. & Lin, S. J. Biol. Chem. 261, 10915-10921 (1986).

1-21. (canceled)

22. A branched polyamine (BPA) having the general following formula (I): 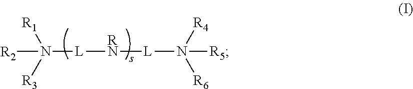
wherein:
each Rᵢ, wherein i is from 1 to 6, is independently selected from the group consisting of a hydrogen atom, and a group of formula (II): 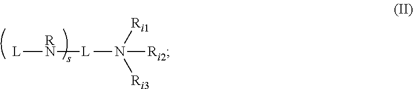
wherein:
each Rᵢⱼ, wherein j is from 1 to 3, is independently selected from the group consisting of a hydrogen atom, and a group of formula (III): 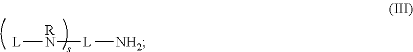
and
each of R₃, R₆ and Rᵢ₃ is independently present or not, if one of R₃, R₆ and Rᵢ₃ is present, the nitrogen atom bearing said group bears a positive charge;
each L is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom;
each R is independently a hydrogen atom or a saturated hydrocarbon group comprising from 1 to 5 carbon atoms;
each s is independently an integer from 0 to 5;
at least 3 groups among R₁, R₂, R₄ and R₅ are different from a hydrogen atom; and optionally at least one of N atom of the groups of formula (II) or (III) is substituted with a label moiety and/or is in the form of an ammonium.

23. The branched polyamine according to claim 22, wherein:
L group depicted in formula (I) is a hydrocarbon group having from 4 to 10 carbon atoms, and/or
L groups depicted in formula (II) or (III) are a hydrocarbon group having from 2 to 6 carbon atoms.

24. The branched polyamine according to claim 22, wherein said branched polyamine is a compound of formula (Ia) or a salt thereof: 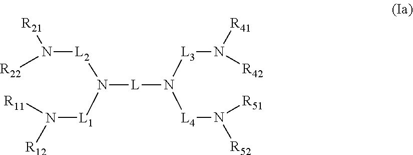
wherein:
L is a saturated or unsaturated C₃-C₁₀ hydrocarbon group,
L₁, L₂, L₃, and L₄ are independently selected from —(CH₂)ₑ— moieties with e being an integer from 2 to 6, and
R₁₁, R₁₂, R₂₁, R₂₂, R₄₁, R₄₂, R₅₁ and R₅₂ are independently selected from the group consisting of H, —Z, —(CH₂)_{f}—NH₂ and —(CH₂)_{f}—NH—Z wherein Z is a label moiety.

25. The branched polyamine according to claim 22, which comprises at least one fluorophore group as a label moiety.

26. The branched polyamine according to claim 22, wherein said branched polyamine is a compound of formula (Ic) or a salt thereof: 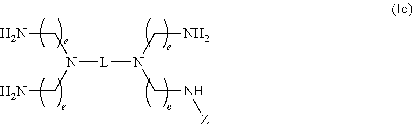
wherein:
e is an integer from 2 to 6,
L is —(CH₂)_{d}— with d being an integer from 3 to 10, and
Z is a fluorophore group selected among cyanine derivatives.

27. The branched polyamine according to claim 22, wherein the polyamine is selected from the group consisting of: 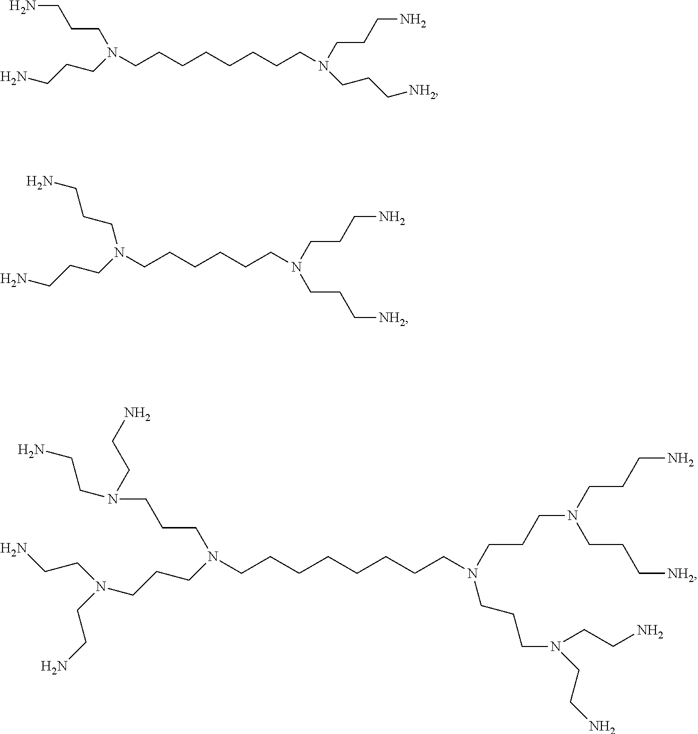 [SMILES: NCCCN(CCCN)CCCCCCCCN(CCCN)CCCN ; NCCCN(CCCN)CCCCCCN(CCCN)CCCN ; NCCCN(CCCN)CCCN(CCCCCCCCN(CCCN(CCN)CCN)CCCN(CCN)CCN)CCCN(CCN)CCN]
and salts thereof.

28. The branched polyamine according to claim 22, wherein said compound is the following:

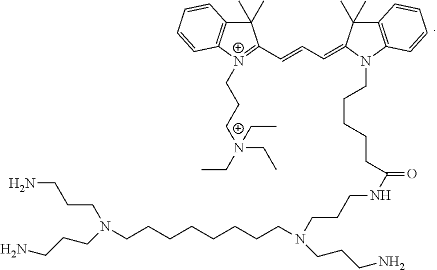

29. A macrocyclic polyamine of formula (V): 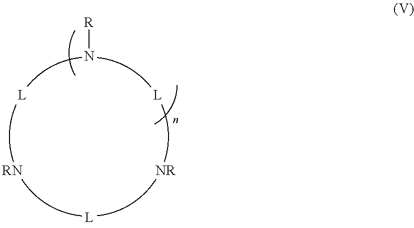
or a salt thereof, wherein:
each L group is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom; and
each R is independently a hydrogen atom, a saturated hydrocarbon group comprising from 1 to 5 carbon atoms or a label moiety; and
n is an integer from 1 to 33; and
wherein the macrocyclic polyamine is different from any one of the compounds of formula (VI): 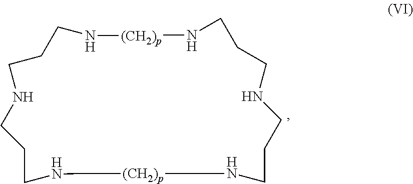
wherein p is 3, 7 or 10.

30. A polyamine derivative comprising at least two polyamine moieties wherein:
the polyamine moieties are independently as defined in claim 22 or formula (V): 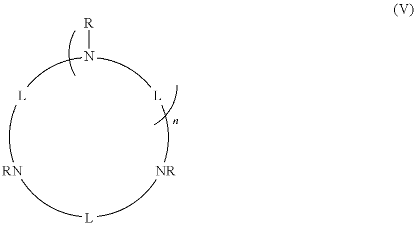
or a salt thereof, wherein:
each L group is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom; and
each R is independently a hydrogen atom, a saturated hydrocarbon group comprising from 1 to 5 carbon atoms or a label moiety; and
n is an integer from 1 to 33; and
wherein the macrocyclic polyamine is different from any one of the compounds of formula (VI): 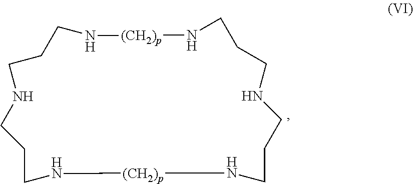
wherein p is 3, 7 or 10, and wherein:
the at least two polyamine moieties being linked by linkers, wherein each linker is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom, or
the polyamine moieties further comprise a core unit to which the at least two polyamine moieties are linked by linkers, wherein each linker is independently a hydrocarbon group selected from the group consisting of: a linear or branched, saturated or unsaturated, hydrocarbon group comprising from 2 to 18 carbon atoms, optionally interrupted by at least one aromatic unit and/or at least one heteroatom such as N, O or S.

31. The polyamine derivative according to claim 30, wherein the core unit comprises an aliphatic cycle or an aromatic cycle, preferably selected among a cycloalkane, a benzene or a naphthalene group.

32. The branched polyamine according to claim 22, wherein at least one nitrogen atom is in the form of an ammonium.

33. A method for impairing a cellular actin-based process, wherein a branched polyamine as defined in claim 22, is implemented, preferably in vitro.

34. A method for impairing a cellular actin-based process, wherein a macrocyclic polyamine of formula (V) or (VI) as defined in claim 29 is implemented, preferably in vitro.

35. A method for impairing a cellular actin-based process, wherein a polyamine derivative as defined in claim 30 is implemented, preferably in vitro

36. A method for promoting lamellipodia growth and/or for slowing-down actin assembly-disassembly dynamics in a cell, wherein a branched polyamine as defined in claim 22 is used, preferably in vitro.

37. A method for promoting lamellipodia growth and/or for slowing-down actin assembly-disassembly dynamics in a cell, wherein a macrocyclic polyamine as defined in claim 29 is used, preferably in vitro.

38. A method for promoting lamellipodia growth and/or for slowing-down actin assembly-disassembly dynamics in a cell, wherein a polyamine derivative as defined in claim 30 is used, preferably in vitro.

39. A method for the treatment of a disease involving protein-protein interaction, protein-nucleic acid interaction and/or nucleic acid-nucleic acid interaction, said disease preferably implying the cytoskeleton, cell migration, cell division, neurodegenerative diseases, such as Alzheimer's disease, diseases implying prions, gene transfer, through interaction with oligo(poly)nucleotide sequences, siRNAs, developmental biology, miRNAs, reparation of cell contacts and cancer in a subject in need of such treatment, wherein it comprises administering to said subject at least one branched polyamine as defined in claim 22.

40. A method for the treatment of a disease involving protein-protein interaction, protein-nucleic acid interaction and/or nucleic acid-nucleic acid interaction, said disease preferably implying the cytoskeleton, cell migration, cell division, neurodegenerative diseases, such as Alzheimer's disease, diseases implying prions, gene transfer, through interaction with oligo(poly)nucleotide sequences, siRNAs, developmental biology, miRNAs, reparation of cell contacts and cancer in a subject in need of such treatment, wherein it comprises administering to said subject at least one macrocyclic polyamine as defined in claim 29.

41. A method for the treatment of a disease involving protein-protein interaction, protein-nucleic acid interaction and/or nucleic acid-nucleic acid interaction, said disease preferably implying the cytoskeleton, cell migration, cell division, neurodegenerative diseases, such as Alzheimer's disease, diseases implying prions, gene transfer, through interaction with oligo(poly)nucleotide sequences, siRNAs, developmental biology, miRNAs, reparation of cell contacts and cancer in a subject in need of such treatment, wherein it comprises administering to said subject at least one polyamine derivative as defined in claim 30.

42. A kit comprising at least one:
a branched polyamine as defined in claim 22,
and optionally monomeric actin and/or actin filaments.

43. The kit according to claim 42, wherein it further comprises at least one additional compound selected in the group consisting of latrunculin A and B, cytochalasin D, jasplakinolide, wiskotatin, CK666, SMIFH2, blebbistatin, ML-7, Y27632, ADF, Arp2/3, an actin nucleation agent such as ActA, IscA, RickA, WASp, N-WASP, pWa and SCAR-WAVE proteins, formins, spire, profilin, gelsolin, capping proteins, a cross-linking protein such as alpha-actinin, fascin, EF-1, Scruin, villin, dematin, fimbrin, spectrin, dystrophin, ABP 120, filamin, and one of their mixtures.

44. The kit of claim 42 which further comprises:
a reagent such as a buffer, culture medium and the like, and/or
a cell or a cell culture, and/or
a device such as microplates, a detection mean and the like, and/or
a detection compound, in particular a compound for the detection of actin structures such as a labeled phalloidin or a labeled antibody directed against actin, and/or
written instructions.

45. A kit comprising at least one:
a macrocyclic polyamine as defined in claim 29,
and optionally monomeric actin and/or actin filaments.

46. A kit comprising at least one:
a polyamine derivative as defined in claim 30,
and optionally monomeric actin and/or actin filaments.

47. An in vitro method for studying assembly-disassembly dynamics of actin filaments or lamellipodium growth in a cell, comprising the steps of:
(a) providing a compound as defined in claim 22,
(b) contacting the compound of step (a) with a cell, and
(c) observing the assembly-disassembly dynamics of actin filaments or lamellipodium growth in said cell.

48. A method for visualizing an actin structure, preferably in a cell, where a compound according to claim 25 is used as an imaging tool.
